SEQUENCE LISTING (12 sequences)

SEQ ID NO: 1 - protein, 118 aa, synthetic construct
  REGION 1..118: note = Synthetic: heavy chain variable region (VH) capable of specifically bind to a human homeobox NKX-2.1
QSLKESEGGLFKPTDTLTLTCTVSGFSLSSYAMIWVRQAPGNGLEYIGAINSYGNAYYASWAKSRSTITRDTNENTVTLKMTSLTAADTATYFCTTTHSYGFGAFDPWGPGTLVTVSS

SEQ ID NO: 2 - protein, 111 aa, synthetic construct
  REGION 1..111: note = Synthetic: light chain variable region (VL) capable of specifically bind to a human homeobox NKX-2.1
AAVLTQTPSPVSAAVGGTVSISCQSSESAYNNRLAWYQQKPGQPPKLLIYDASKLASGVPSRFKGSGSGTPFTLTISGVQCADAATYYCQGVYSSVSDVYAFGGGTEVVVK

SEQ ID NO: 3 - protein, 323 aa, synthetic construct
  REGION 1..323: note = Synthetic: heavy chain constant region
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 4 - protein, 441 aa, synthetic construct
  REGION 1..441: note = Synthetic: heavy chain
QSLKESEGGLFKPTDTLTLTCTVSGFSLSSYAMIWVRQAPGNGLEYIGAINSYGNAYYASWAKSRSTITRDTNENTVTLKMTSLTAADTATYFCTTTHSYGFGAFDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 5 - protein, 19 aa, synthetic construct
  REGION 1..19: note = Synthetic: terminal signal peptide
METGLRWLLLVAVLKGVQC

SEQ ID NO: 6 - protein, 137 aa, synthetic construct
  REGION 1..137: note = Synthetic: heavy chain variable region signal peptide
METGLRWLLLVAVLKGVQCQSLKESEGGLFKPTDTLTLTCTVSGFSLSSYAMIWVRQAPGNGLEYIGAINSYGNAYYASWAKSRSTITRDTNENTVTLKMTSLTAADTATYFCTTTHSYGFGAFDPWGPGTLVTVSS

SEQ ID NO: 7 - protein, 460 aa, synthetic construct
  REGION 1..460: note = Synthetic: heavy chain variable plus constant region, with signal peptide
METGLRWLLLVAVLKGVQCQSLKESEGGLFKPTDTLTLTCTVSGFSLSSYAMIWVRQAPGNGLEYIGAINSYGNAYYASWAKSRSTITRDTNENTVTLKMTSLTAADTATYFCTTTHSYGFGAFDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 8 - protein, 104 aa, synthetic construct
  REGION 1..104: note = Synthetic: light chain constant region
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 9 - protein, 215 aa, synthetic construct
  REGION 1..215: note = Synthetic: light chain
AAVLTQTPSPVSAAVGGTVSISCQSSESAYNNRLAWYQQKPGQPPKLLIYDASKLASGVPSRFKGSGSGTPFTLTISGVQCADAATYYCQGVYSSVSDVYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 10 - protein, 133 aa, synthetic construct
  REGION 1..133: note = Synthetic: light chain variable region with a signal peptide
MDTRAPTQLLGLLLLWLPGATFAAVLTQTPSPVSAAVGGTVSISCQSSESAYNNRLAWYQQKPGQPPKLLIYDASKLASGVPSRFKGSGSGTPFTLTISGVQCADAATYYCQGVYSSVSDVYAFGGGTEVVVK

SEQ ID NO: 11 - protein, 237 aa, synthetic construct
  REGION 1..237: note = Synthetic: light chain variable and constant region with a signal peptide
MDTRAPTQLLGLLLLWLPGATFAAVLTQTPSPVSAAVGGTVSISCQSSESAYNNRLAWYQQKPGQPPKLLIYDASKLASGVPSRFKGSGSGTPFTLTISGVQCADAATYYCQGVYSSVSDVYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 12 - protein, 22 aa, synthetic construct
  REGION 1..22: note = Synthetic: amino terminal signal peptide
MDTRAPTQLLGLLLLWLPGATF

(19) United States
(12) Patent Application Publication
(10) Pub. No.: US 2026/0285955 A1
(43) Pub. Date: Sep. 24, 2026

(54) ANTI-HUMAN HOMEOBOX PROTEIN NKX-2.1 ANTIBODIES FOR USE IN IMMUNOHISTOCHEMISTRY (IHC) PROTOCOLS AND FOR DIAGNOSING CANCER

(71) Applicant: Agilent Technologies, Inc., Santa Clara, CA (US)

(72) Inventors: Morten Draeby SORENSEN, Herlev (DK); Tine HAGEDORN-OLSEN, Skibby (DK); Lasse RAMSGAARD, Glostrup (DK); Morten Alder SCHULZ, Bagsvaerd (DK)

(21) Appl. No.: 19/112,467

(22) PCT Filed: Sep. 27, 2023
(86) PCT No.: PCT/US2023/033881

§ 371 Date: Mar. 17, 2025

Related U.S. Application Data
(60) Provisional application No. 63/411,913, filed on Sep. 30, 2022.

(51) Int. Cl. C07K 16/18; G01N 33/575; G01N 33/5752

(52) U.S. Cl. CPC C07K 16/18; G01N 33/5752; G01N 33/57557; G01N 33/57595; C07K 2317/33; C07K 2317/52; C07K 2317/565; C07K 2319/02; G01N 2333/4703; G01N 2474/20

(57) ABSTRACT

In alternative embodiments, provided are chimeric, synthetic or recombinant anti-human homeobox NKX-2.1 (also called NK2 homeobox 1, or thyroid transcription factor 1 (TTF-1)) protein antibodies (Abs), including products of manufacture and kits comprising them, and methods for making and using them, including for example their use in the detection or diagnosis, and treatment, of a cancer. In alternative embodiments, anti-NKX-2.1 (TTF-1) antibodies as provided herein are used together with an agent for determining whether NKX-2.1 (TTF-1) expression or activity is present, reduced or absent; or an Ab, or antigen (Ag) binding fragment or protein (ABP) thereof, or monomeric or dimeric ABP, as provided herein is less sensitive (or binds with lower affinity) in specific binding to a lung squamous cell carcinoma cell than specifically binding to an adenocarcinoma cell, thereby resulting in fewer lung squamous cell carcinoma false positives and better positive identification of lung adenocarcinoma.

### RELATED APPLICATIONS

This application is the 35 U.S.C. § 371 application corresponding with the PCT international utility patent application PCT/US2023/033881 filed Sep. 27, 2023 and which claims the benefit of priority under 35 U.S.C. § 119(e) to U.S. Provisional Patent Application Ser. No. 63/411,913, Sep. 30, 2022. The aforementioned application is expressly incorporated herein by reference in its entirety and for all purposes.

### REFERENCE TO ELECTRONIC SEQUENCE LISTING

The application contains a Sequence Listing which has been submitted electronically in XML format and is hereby incorporated by reference in its entirety. Said XML copy, created on Nov. 29, 2023, is named “6363.145313PCT.xml” and is 18,767 bytes in size. The sequence listing contained in this XML file is part of the specification and is hereby incorporated by reference herein in its entirety.

### TECHNICAL FIELD

This invention generally relates to immunohistochemistry (IHC) and cancer diagnosis and treatment. In alternative embodiments, provided are chimeric, synthetic or recombinant anti-human homeobox NKX-2.1 (also called NK2 homeobox 1, or thyroid transcription factor 1 (TTF-1)) protein antibodies (Abs), including products of manufacture and kits comprising them, and methods for making and using them, including for example their use in the detection or diagnosis, and treatment, of a cancer, or other diseases or conditions involving expression of TTF-1. In alternative embodiments, anti-NKX-2.1 (TTF-1) antibodies as provided herein are used together with an agent for determining whether NKX-2.1 (TTF-1) expression or activity is present, reduced or absent. In alternative embodiments, antibodies (Ab) as provided herein may be able to help to distinguish adenocarcinoma from squamous cell carcinoma in non-small cell lung cancer; an Ab, or antigen (Ag) binding fragment or protein (ABP) thereof, or monomeric or dimeric ABP, as provided herein is less sensitive (or binds with lower affinity) in specific binding to a lung squamous cell carcinoma cell than specifically binding to an adenocarcinoma cell, thereby resulting in fewer lung squamous cell carcinoma false positives and better positive identification of lung adenocarcinoma.

### BACKGROUND

Thyroid transcription factor-1 (TTF-1), or homeobox NKX-2.1 (also called NK2 homeobox 1), belongs to a family of homeodomain transcription factors and is selectively expressed in thyroid, lung, and diencephalon. TTF-1 has been identified as a transcriptional regulator of thyroid specific genes and has also been shown to be important in the activation of pulmonary-specific differentiation genes (4, 5).

While known anti-human TTF-1 antibodies can be used in the identification and diagnosis of certain cancers, currently used anti-human TTF-1 antibodies may not be able to distinguish a lung adenocarcinoma from a squamous cell carcinoma with high sensitivity and/or specificity.

Alternative names (synonyms) for homeobox protein Nkx-2.1, gene: NKX2-1, are: Homeobox protein NK-2 homolog A; Thyroid nuclear factor 1; Thyroid transcription factor 1, short name “TTF-1”; thyroid-specific enhancer-binding protein, short name T/EBP.

### SUMMARY

In alternative embodiments, provided are isolated or purified antibodies (Ab), or synthetic or recombinant Ab, or antigen (Ag) binding fragment thereof, or monomeric or dimeric antigen binding protein (ABP), capable of specifically binding a human homeobox NKX-2.1 (also called NK2 homeobox 1, or thyroid transcription factor 1 (TTF-1)) protein or polypeptide,

wherein the isolated or purified Ab, or synthetic or recombinant Ab, or Ag binding fragment thereof, or monomeric or dimeric ABP comprises:
(a) a heavy chain variable region (VH) capable of specifically binding to a human homeobox NKX-2.1 protein or polypeptide, comprising:
   (1) an amino acid sequence comprising the three complementarity determining regions (CDRs) CDR1, CDR2 and CDR3 of SEQ ID NO:1, or CDR1 amino acid (aa) residues GFSLSSYA (residues 25 to 32 of SEQ ID NO:1), CDR2 aa residues INSYGNA (residues 50 to 56 of SEQ ID NO:1), and CDR3 aa residues TTTHSYGFGAFDP (residues 95 to 107 of SEQ ID NO:1), or
   (2) amino acid sequences having at least about 70%, 75%, 80%, 85%, 90%, 95%, 98% sequence identity, or between about 70% to 100% sequence identity, to each of the three complementarity determining regions (CDRs) CDR1, CDR2 and CDR3 of SEQ ID NO:1, or CDR1 amino acid (aa) residues GFSLSSYA (residues 25 to 32 of SEQ ID NO:1), CDR2 aa residues INSYGNA (residues 50 to 56 of SEQ ID NO:1), and CDR3 aa residues TTTHSYGFGAFDP (residues 95 to 107 of SEQ ID NO:1),
   and optionally the heavy chain variable region (VH) capable of specifically bind to a human homeobox NKX-2.1 protein or polypeptide is, or comprises an amino acid sequence:

(SEQ ID NO: 1)
QSLKESEGGLFKPTDTLTLTCTVSGFSLSSYAMIWVRQAPGNGLEYIG
AINSYGNAYYASWAKSRSTITRDTNENTVTLKMTSLTAADTATYF
CTTTHSYGFGAFDPWGPGTLVTVSS;

(3) an amino acid sequence having at least about 70%, 75%, 80%, 85%, 90%, 95%, 98% sequence identity, or between about 70% to 100% sequence identity, to SEQ ID NO:1, or an amino acid sequence having complete sequence identity to SEQ ID NO:1;
(b) a light chain variable region (VL) capable of specifically bind to a human homeobox NKX-2.1 protein or polypeptide comprising:
   (1) an amino acid sequence comprising the three CDRs: CDR1, CDR2 and CDR3 of SEQ ID NO:2, or CDR1 amino acid (aa) residues ESAYNNR (residues 27 to 33 of SEQ ID NO:2), CDR2 aa residues DAS (residues 51 to 53 of SEQ ID NO:2), and CDR3 aa residues QGVYSSVSDVYA (residues 112 to 123 of SEQ ID NO:2), or
   (2) amino acid sequences having at least about 70%, 75%, 80%, 85%, 90%, 95%, 98% sequence identity, or between about 70% to 100% sequence identity, to each of the three CDRs: CDR1, CDR2 and CDR3 of SEQ ID NO:2, or CDR1 amino acid (aa) residues ESAYNNR (residues 27 to 33 of SEQ ID NO:2), CDR2 aa residues DAS (residues 51 to 53 of SEQ ID NO:2), and CDR3 aa residues QGVYSSVSDVYA (residues 112 to 123 of SEQ ID NO:2),
   wherein optionally the light chain variable region (VL) capable of specifically bind to a human homeobox NKX-2.1 protein or polypeptide comprises or is an amino acid sequence:

(SEQ ID NO: 2)
AAVLTQTPSPVSAAVGGTVSISCQSSESAYNNRLAWYQQKPGQPPKLL
IYDASKLASGVPSRFKGSGSGTPFTLTISGVQCADAATYYCQGVYSSV
SDVYAFGGGTEVVVK;

(3) an amino acid sequence having at least about 70%, 75%, 80%, 85%, 90%, 95%, 98% sequence identity, or between about 70% to 100% sequence identity, to SEQ ID NO:2, or an amino acid sequence having complete (100%) sequence identity to SEQ ID NO:2; or
(c) a heterodimer capable of specifically binding to a human homeobox NKX-2.1 protein or polypeptide comprising the heavy chain variable region (VH) of (a) and the light chain variable region (VL) of (b).

In alternative embodiments, the isolated or purified Ab, or synthetic or recombinant Ab, or Ag binding fragment thereof, or monomeric or dimeric ABP, as provided herein, are fabricated as or are in the form of:
an antigen-binding fragment, wherein optionally the antigen-binding fragment comprises a Fab, or an Ab fragment having at least one one constant and one variable domain of each of an Ab heavy chain and an Ab light chain,
a F(ab′)₂ (or an Ab digested by pepsin yielding two fragments: a F(ab′)₂ fragment and a pFc′ (pepsin cleavage Fc) fragment),
   a Fab′ (a single chain of a F(ab′)₂ fragment),
   a single-chain variable fragment (scFv) (or a fusion protein of a variable region of an Ab heavy and light chain connected with a linker peptide optionally of about ten to about 25 amino acids in length),
   a (scFv)₂, or a di-scFv or a bi-scFv, or a single peptide chain having two variable heavy and two variable light regions yielding tandem scFv,
   a minibody, or a fusion protein comprising a variable region of an Ab heavy and a variable region of a light chain connected with or linked an alkyl group, wherein optionally the alkyl group comprises a methyl or an ethyl group,
   a diabody (or an scFv with a linker peptide too short (optionally about five amino acids) for the two variable regions to fold together forcing the scFvs to dimerize), a triabody or a tetrabody (or an scFv with a linker peptide too short (optionally about one or two amino acids) for the two variable regions to fold together forcing the scFvs to trimerize or tetramize),
   a single-domain antibody (dAB) (or a single variable region of an Ab heavy or Ab light chain),
   a plurality of complementarity determining region (CDR) fragments, or
   a multispecific antibody formed from two or more antibody fragments.

In alternative embodiments of isolated or purified Ab, or synthetic or recombinant Ab, or Ag binding fragment thereof, or monomeric or dimeric ABP, as provided herein:
the heavy chain variable region (VH), if present:
   (a) further comprises a carboxy terminal constant domain, or further comprises amino acid sequence SEQ ID NO:3, or comprises SEQ ID NO:4;
   (b) further comprises an amino terminal signal peptide, or further comprises an amino terminal signal peptide comprising: METGLRWLLLVAVLKGVQC (SEQ ID NO:5), or comprises:
   a heavy chain variable region having a signal peptide comprising an amino acid sequence:

(SEQ ID NO: 6)
METGLRWLLLVAVLKGVQCQSLKESEGGLFKPTDTLTLTCTVSGFS
LSSYAMIWVRQAPGNGLEYIGAINSYGNAYYASWAKSRSTITRDTN
ENTVTLKMTSLTAADTATYFCTTTHSYGFGAFDPWGPGTLVTVSS,

or
a heavy chain variable plus constant region, with signal peptide, comprising an amino acid sequence:

(SEQ ID NO: 7)
METGLRWLLLVAVLKGVQCQSLKESEGGLFKPTDTLTLTCTVSGFSL
SSYAMIWVRQAPGNGLEYIGAINSYGNAYYASWAKSRSTITRDTNEN
TVTLKMTSLTAADTATYFCTTTHSYGFGAFDPWGPGTLVTVSS
GQPKAPSVFPLAPCCGDTPSSTVTLGCLVKGYLPEPVTVTWNSGTLT
NGVRTFPSVRQSSGLYSLSSVVSVTSSSQPVTCNVAHPATNTKVDKT
VAPSTCSKPTCPPPELLGGPSVFIFPPKPKDTLMISRTPEVTCVVVDVS
QDDPEVQFTWYINNEQVRTARPPLREQQFNSTIRVVSTLPIAHQDWLR
GKEFKCKVHNKALPAPIEKTISKARGQPLEPKVYTMGPPREELSSRSV
SLTCMINGFYPSDISVEWEKNGKAEDNYKTTPAVLDSDGSYFLYSKL
SVPTSEWQRGDVFTCSVMHEALHNHYTQKSISRSPGK;

or
   (c) comprises SEQ ID NO:4, SEQ ID NO:6 or SEQ ID NO:7, having one or more amino acid substitutions, additions (insertions) or deletions, and the recombinant Ab, or Ag binding fragment thereof, or monomeric or dimeric ABP retains its ability to specifically bind to a human homeobox NKX-2.1 protein or polypeptide,

and optionally the one or more amino acid substitutions comprise one or more conservative amino acid substitutions, and the isolated or purified Ab, or synthetic or recombinant Ab, or Ag binding fragment thereof, or monomeric or dimeric ABP, retains its ability to specifically bind to a human homeobox NKX-2.1 protein or polypeptide.

In alternative embodiments of isolated or purified Ab, or synthetic or recombinant Ab, or Ag binding fragment thereof, or monomeric or dimeric ABP, as provided herein: the light chain variable region (VL), if present:
(a) further comprises a carboxy terminal constant domain, or further comprises amino acid sequence SEQ ID NO:8, or comprises SEQ ID NO:9,
(b) further comprises an amino terminal signal peptide, or further comprises an amino terminal signal peptide consisting of MDTRAPTQLLGLLLLWLPGATF (SEQ ID NO:12), or comprises:
a light chain variable region with a signal peptide having an amino acid sequence:

(SEQ ID NO: 10)
MDTRAPTQLLGLLLLWLPGATFAAVLTQTPSPVSAAVGGTVSISCQS
SESAYNNRLAWYQQKPGQPPKLLIYDASKLASGVPSRFKGSGSG
TPFTLTISGVQCADAATYYCQGVYSSVSDVYAFGGGTEVVVK,

or
a light chain variable and constant region with a signal peptide having an amino acid sequence:

(SEQ ID NO: 11)
MDTRAPTQLLGLLLLWLPGATFAAVLTQTPSPVSAAVGGTVSISCQSS
ESAYNNRLAWYQQKPGQPPKLLIYDASKLASGVPSRFKGSGSG
TPFTLTISGVQCADAATYYCQGVYSSVSDVYAFGGGTEVVVKGDPV
APTVLIFPPAADQVATGTVTIVCVANKYFPDVTVTWEVDGTTQTT
GIENSKTPQNSADCTYNLSSTLTLTSTQYNSHKEYTCKVTQGTTSVVQ
SFNRGDC;

or
(c) comprises SEQ ID NO:9, SEQ ID NO:10 or SEQ ID NO:11, having one or more amino acid substitutions, additions (insertions) or deletions, and the recombinant Ab, or Ag binding fragment thereof, or monomeric or dimeric ABP retains its ability to specifically bind to a human homeobox NKX-2.1 protein or polypeptide,

and optionally the one or more amino acid substitutions comprise one or more conservative amino acid substitutions, and the isolated or purified Ab, or synthetic or recombinant Ab, or Ag binding fragment thereof, or monomeric or dimeric ABP, retains its ability to specifically bind to a human homeobox NKX-2.1 protein or polypeptide.

In alternative embodiments: the one or more amino acid substitutions comprise having two, three, four, five, six, seven, eight, nine, ten, eleven, twelve thirteen, fourteen or fifteen conservative amino acid substitutions, and the recombinant Ab, or Ag binding fragment thereof, or monomeric or dimeric ABP retains its ability to specifically bind to a human homeobox NKX-2.1 protein or polypeptide.

In alternative embodiments of isolated or purified Ab, or synthetic or recombinant Ab, or Ag binding fragment thereof, or monomeric or dimeric ABP, as provided herein:
the light chain variable region further comprises at least a portion of a light chain constant region;
the light chain constant region comprises an amino acid sequence:

(SEQ ID NO: 8)
GDPVAPTVLIFPPAADQVATGTVTIVCVANKYFPDVTVTWEVDGTTQTT
GIENSKTPQNSADCTYNLSSTLTLTSTQYNSHKEYTCKVTQGTTSVVQS
FNRGDC;

the heavy chain variable region further comprises at least a portion of a heavy chain constant region;
the heavy chain constant region comprises an amino acid sequence:

(SEQ ID NO: 3)
GQPKAPSVFPLAPCCGDTPSSTVTLGCLVKGYLPEPVTVTWNSGTLTNG
VRTFPSVRQSSGLYSLSSVVSVTSSSQPVTCNVAHPATNTKVDKTVAPS
TCSKPTCPPPELLGGPSVFIFPPKPKDTLMISRTPEVTCVVVDVSQDDP
EVQFTWYINNEQVRTARPPLREQQFNSTIRVVSTLPIAHQDWLRGKEFK
CKVHNKALPAPIEKTISKARGQPLEPKVYTMGPPREELSSRSVSLTCMI
NGFYPSDISVEWEKNGKAEDNYKTTPAVLDSDGSYFLYSKLSVPTSEWQ
RGDVFTCSVMHEALHNHYTQKSISRSPGK;

the light chain variable region further comprises at least a portion of a light chain constant region; and, the heavy chain variable region further comprises at least a portion of a heavy chain constant region;
the isolated or purified, or the recombinant antibody (Ab), or antigen binding fragment thereof, or monomeric or dimeric antigen binding protein, comprises:

a light chain comprising an amino acid sequence (SEQ ID NO:9):

(SEQ ID NO: 9)
AAVLTQTPSPVSAAVGGTVSISCQSSESAYNNRLAWYQQKPGQPPKL
LIYDASKLASGVPSRFKGSGSGTPFTLTISGVQCADAATYYCQGVYS
SVSDVYAFGGGTEVVVKGDPVAPTVLIFPPAADQVATGTVTIVCVA
NKYFPDVTVTWEVDGTTQTTGIENSKTPQNSADCTYNLSSTLTLTST
QYNSHKEYTCKVTQGTTSVVQSFNRGDC,

a heavy chain comprising an amino acid sequence (SEQ ID NO:4):

(SEQ ID NO: 4)
QSLKESEGGLFKPTDTLTLTCTVSGFSLSSYAMIWVRQAPGNGLEYIGA
INSYGNAYYASWAKSRSTITRDTNENTVTLKMTSLTAADTATYFCTTTH
SYGFGAFDPWGPGTLVTVSSGQPKAPSVFPLAPCCGDTPSSTVTLGCLV
KGYLPEPVTVTWNSGTLTNGVRTFPSVRQSSGLYSLSSVVSVTSSSQPV
TCNVAHPATNTKVDKTVAPSTCSKPTCPPPELLGGPSVFIFPPKPKDTL
MISRTPEVTCVVVDVSQDDPEVQFTWYINNEQVRTARPPLREQQFNSTI
RVVSTLPIAHQDWLRGKEFKCKVHNKALPAPIEKTISKARGQPLEPKVY
TMGPPREELSSRSVSLTCMINGFYPSDISVEWEKNGKAEDNYKTTPAVL
DSDGSYFLYSKLSVPTSEWQRGDVFTCSVMHEALHNHYTQKSISRSPG
K;

the heavy chain constant region comprises amino acid sequence from a IgG, IgM, IgA, IgD or IgE isotype, or the heavy chain constant region substantially comprises amino acid sequence from a IgG, IgM, IgA, IgD or IgE isotype;
the light chain constant region comprises amino acid sequence from a kappa (κ) or lambda (λ) isotype, or the light chain constant region substantially comprises amino acid sequence from a kappa (κ) or lambda (λ) isotype;
the at least a portion of the heavy chain constant region, at least a portion of the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat or substantially derived from a human, a rabbit, a mouse or a rat;
at least a portion of the heavy chain constant region, at least a portion of the light chain constant region, or at least a portion of the heavy chain constant region and the light chain constant region, is or comprises a synthetic amino acid sequence;
the recombinant Ab, the Ag binding fragment thereof, or monomeric or dimeric ABP, or the heavy chain constant region, or the light chain constant region, or the heavy chain constant region and the light chain constant region, further comprises or is bound to a heterologous protein, peptide, or a compound or a composition;
the heterologous protein or peptide, or the compound or a composition, comprises a detectable protein, a detectable agent or a binding moiety, or the heterologous protein or peptide comprises a carrier protein;
the heterologous protein, peptide or the compound or composition, is covalently conjugated to the recombinant antibody (Ab), or Ag binding fragment thereof, or monomeric or dimeric ABP;
the detectable agent or binding moiety comprises a biotin, a fluorescent or chemiluminescent label, a fluorophore, perylene, fluorenyl, coumarin, 7-methoxycoumarin (Mca), 4-(dimethylaminoazo)benzene-4-carboxylic acid (dabcyl), Tamra, boron-dipyrromethene (BODIPY), or derivatives thereof, a dye, a radioisotope, a quantum dot or photoluminescent aqueous nanocrystal, a hapten, or an antibody binding epitope or domain, and optionally the dye is or comprises rhodamine, [2-(4-nitro-2,1,3-benzoxadiazol-7-yl)aminoethyl]trimethylammonium (NBD), nile red or nile blue, or is a fluorescent dye comprising sulfoindocyanine, and optionally the fluorophore is or comprises a dansyl, a fluorescein, a carboxyfluorescein (FAM) or a 6-FAM moiety, and optionally the dye is or comprises a cyanine dye, a Cy3 or a Cy5, and optionally the hapten is or comprises a biotin, a theophylline, a digoxigenin, a carborane, a fluorescein or a bromodeoxyuridine moiety; and/or
the Ab, or Ag binding fragment thereof, or monomeric or dimeric ABP is a recombinant Ab, or Ag binding fragment thereof, or monomeric or dimeric ABP, or comprises a peptide or polypeptide made by a recombinant technique.

In alternative embodiments, provided are chimeric, synthetic or recombinant nucleic acids comprising: a nucleic acid sequence encoding a Ab, or Ag binding fragment thereof, or monomeric or dimeric ABP as provided herein.

In alternative embodiments of the chimeric, synthetic or recombinant nucleic acids as provided herein:
the chimeric or recombinant nucleic acid further comprises and is operatively linked to a transcriptional regulatory element, and optionally the transcriptional regulatory element comprises a promoter, and optionally the promoter is an inducible promoter or a constitutive promoter; and/or
the chimeric or recombinant nucleic acid further comprises sequence encoding an amino terminal signal peptide, and optionally the amino terminal signal peptide comprises the amino acid sequence: METGLRWLLLVAVLKGVQC (SEQ ID NO:5); MDTRAPTQLLGLLLLWLPGATF (SEQ ID NO:12), and optionally the amino terminal signal peptide SEQ ID NO:5 is amino terminal to a VH chain SEQ ID NO:1, or is amino terminal to SEQ ID NO:4; or the amino terminal signal peptide SEQ ID NO:12 is amino terminal to a VL chain SEQ ID NO:2, or is amino terminal to SEQ ID NO:9.

In alternative embodiments, provided are expression cassettes, vectors, recombinant viruses, artificial chromosomes, cosmids or plasmids comprising a chimeric, synthetic or a recombinant nucleic acid as provided herein.

In alternative embodiments, provided are cells comprising a chimeric, synthetic or recombinant antibody or dimeric antigen binding protein as provided herein, a chimeric or recombinant nucleic acid as provided herein, or an expression cassette, vector, recombinant virus, artificial chromosome, cosmid or plasmid as provided herein. The cell can be a bacterial, fungal, mammalian, yeast, insect or plant cell, or a mammalian cell or a human cell.

In alternative embodiments, provided are methods for detecting the presence of a human homeobox NKX-2.1 protein or polypeptide in or on a cell, a tissue, an organ or a portion of any of the foregoing, comprising:
(a) contacting the cell, tissue or organ or portion of any of the foregoing with at least one isolated or purified, or synthetic or recombinant Ab, or Ag binding fragment thereof, or monomeric or dimeric ABP as provided herein, and
(b) detecting the specific binding of the at least one Ab, or Ag binding fragment thereof, or monomeric or dimeric ABP with a human homeobox NKX-2.1 protein or polypeptide, in or on the cell, tissue or organ or portion of any of the foregoing, thereby detecting the presence of the human homeobox NKX-2.1 protein or polypeptide in or on the cell, tissue, organ or portion of any of the foregoing.

In alternative embodiments of methods for detecting the presence of a human homeobox NKX-2.1 protein or polypeptide as provided herein:
the at least one Ab, or Ag binding fragment thereof, or monomeric or dimeric ABP comprises a variable heavy chain (VH) having an amino acid sequence comprising SEQ ID NO:1 and a variable light chain (VL) having an amino acid sequence comprising SEQ ID NO:2;
the at least one Ab, or Ag binding fragment thereof, or monomeric or dimeric ABP comprises a variable heavy chain (VH) having an amino acid sequence comprising SEQ ID NO:4 and a variable light chain (VL) having an amino acid sequence comprising SEQ ID NO:9;
the at least one Ab, or Ag binding fragment thereof, or monomeric or dimeric ABP comprises a variable heavy chain (VH) having an amino acid sequence comprising SEQ ID NO:7 and a variable light chain (VL) having an amino acid sequence comprising SEQ ID NO:11;
the method comprises contacting the cell, tissue or organ or portion of any of the foregoing with two or at least two Abs, or Ag binding fragments thereof, or monomeric or dimeric ABPs, or a mixture of two or at least two different Abs, or Ag binding fragments thereof, or monomeric or dimeric ABPs, wherein the two or at least two Abs, or Ag binding fragments thereof, or monomeric or dimeric ABPs or the two or at least two different Abs, or Ag binding fragments thereof, or monomeric or dimeric ABPs, comprise an Abs, or Ag binding fragments thereof, or monomeric or dimeric ABPs as provided herein;
the contacting comprises use of an immunohistochemistry (IHC) assay;
the method further comprises contacting the Ab, or Ag binding fragment thereof, or monomeric or dimeric ABP, specifically bound to a human homeobox NKX-2.1 polypeptide or peptide, with a detectable agent to indicate or signal the specific binding of the Ab, or Ag binding fragment thereof, or monomeric or dimeric ABP, to the human homeobox NKX-2.1 polypeptide or peptide, and optionally the detectable agent specifically binds to the Ab, or Ag binding fragment thereof, or monomeric or dimeric ABP;
the cell, tissue, organ or a portion of any of the foregoing is or comprises or is derived from:

a cancer cell,
wherein optionally the cancer cell is a thyroid cancer cell, a lung cancer cell, or a carcinoma cell,
wherein optionally the carcinoma cell is a large cell carcinoma, a small cell carcinoma (optionally a small cell lung carcinoma or an extrapulmonary small cell carcinoma), a lung carcinoma cell or an adenocarcinoma cell, and optionally the adenocarcinoma cell is a pulmonary adenocarcinoma cell or a thyroid adenocarcinoma cell),
wherein optionally the cancer cell is a squamous carcinoma (SCC) cell, a leukemia cell, a carcinoid tumor, a mamma carcinoma, a colon carcinoma cell, a malignant melanoma cell or a multiple myeloma cell, a plasmacytoma cell or a lymphoma cell or a cancer derived from a lymphocyte,
   wherein optionally the lymphocyte is an early B cell, a pro-B cell, a pre-B lymphocyte, a mature B-lymphocyte, a T lymphocyte, a parafollicular T lymphocyte, a cell derived from a subpopulation of parafollicular T lymphocytes,
a lung cell, wherein optionally the lung cell is a lung pneumocyte cell (optionally a type II pneumocyte cell) or a Clara (or nonciliated bronchiolar secretory) cell,
a thyroid cell, optionally a thyroid follicular cell or a thyroid parafollicular cell,
a follicular center cell, or a cell in a tonsil, a lymph node germinal center, a bone marrow stem cell, a myelopoietic cell, a liver bile canalicular cell, a renal glomerular cell, a proximal tubular cell, a breast myoepithelial cell, a stromal cell around or associated with an infiltrating tumor cell, a kidney cell, an epithelial cell, a cerebellum cell, a prostate cell, a kidney cell, a pancreas cell, a bone marrow cell,
   and optionally the epithelial cell is or is derived from a brain, lung, intestine, kidney, breast or placental epithelial cell, and optionally the organ is a liver, prostate or lung; and/or
the Ab, or Ag binding fragment thereof, or monomeric or dimeric ABP is less sensitive (or binds with lower affinity) in specific binding to lung squamous cell carcinoma cells than specifically binding to adenocarcinoma cells, thereby resulting in fewer lung squamous cell carcinoma false positives and better positive identification of lung adenocarcinoma.

In alternative embodiments, provided are methods for detecting or diagnosing a cancer,

wherein the method comprises detecting expression or presence of a human homeobox NKX-2.1 polypeptide or peptide in or on a cell, tissue or organ sample using a method as provided herein,

wherein the detecting of the specific binding of the Ab, or Ag binding fragment thereof, or monomeric or dimeric ABP with a human homeobox NKX-2.1 polypeptide or peptide in or on the cell, tissue or organ or portion of any of the foregoing, detects or diagnoses, or assists in the detection or diagnosis of, the cancer.

In alternative embodiments of methods for detecting or diagnosing a cancer as provided herein:
the cancer is:
   a thyroid cancer cell, a lung cancer cell, or a carcinoma cell,
   wherein optionally the carcinoma cell is a large cell carcinoma, a small cell carcinoma (optionally a small cell lung carcinoma or an extrapulmonary small cell carcinoma), a lung carcinoma cell or an adenocarcinoma cell, and optionally the adenocarcinoma cell is a pulmonary adenocarcinoma cell or a thyroid adenocarcinoma cell),
   wherein optionally the cancer cell is a squamous carcinoma cell (SCC), a leukemia cell, a carcinoid tumor, a mamma carcinoma, a colon carcinoma cell, a malignant melanoma cell or a multiple myeloma cell, a plasmacytoma cell or a lymphoma cell or a cancer derived from a lymphocyte,
      wherein optionally the lymphocyte is an early B cell, a pro-B cell, a pre-B lymphocyte, a mature B-lymphocyte, a T lymphocyte, a parafollicular T lymphocyte, a cell derived from a subpopulation of parafollicular T lymphocytes;
the Ab, or Ag binding fragment thereof, or monomeric or dimeric ABP is less sensitive (or binds with lower affinity) in specific binding to lung squamous cell carcinoma cells than specifically binding to adenocarcinoma cells, thereby resulting in fewer lung squamous cell carcinoma false positives and better positive identification of lung adenocarcinoma;
the cell, tissue or organ sample is from an individual in need thereof;
the detection comprises conducting an immunohistochemistry (IHC) assay;
at least two Abs, or Ag binding fragments thereof, or monomeric or dimeric ABPs, are used to contact the cell, tissue or organ sample; and/or
one of the two Abs, or Ag binding fragments thereof, or monomeric or dimeric ABPs comprises a variable heavy chain (VH) having an amino acid sequence comprising SEQ ID NO:1 and a variable light chain (VL) having an amino acid sequence comprising SEQ ID NO:2.

In alternative embodiments, provided are methods for treating, ameliorating or preventing (for example, further growth or spread of) a cancer comprising first detecting or diagnosing the cancer using a method as provided herein, followed by treatment of (or treating, or ameliorating) the individual in need thereof for the cancer.

In alternative embodiments of methods for treating, ameliorating or preventing a cancer as provided herein:
the cancer is:
   a thyroid cancer, a lung cancer, or a carcinoma,
   wherein optionally the carcinoma is a large cell carcinoma, a small cell carcinoma (optionally a small cell lung carcinoma or an extrapulmonary small cell carcinoma), a lung carcinoma or an adenocarcinoma, and optionally the adenocarcinoma is a pulmonary adenocarcinoma or a thyroid adenocarcinoma),
   wherein optionally the cancer is a squamous carcinoma cell (SCC), a leukemia, a carcinoid tumor, a mamma carcinoma, a colon carcinoma cell, a malignant melanoma or a multiple myeloma, a plasmacytoma or a lymphoma or a cancer derived from a lymphocyte,
      wherein optionally the lymphocyte is an early B cell, a pro-B cell, a pre-B lymphocyte, a mature B-lymphocyte, a T lymphocyte, a parafollicular T lymphocyte, a cell derived from a subpopulation of parafollicular T lymphocytes; and/or
the Ab, or Ag binding fragment thereof, or monomeric or dimeric ABP is less sensitive (or binds with lower affinity) in specific binding to lung squamous cell carcinoma cells than specifically binding to adenocarcinoma cells, thereby resulting in fewer lung squamous cell carcinoma false positives and better positive identification of lung adenocarcinoma.

In alternative embodiments, provided are uses of at least one isolated or purified, or synthetic or recombinant antibody (Ab), or antigen (Ag) binding fragment thereof, or monomeric or dimeric antigen binding protein (ABP) as provided herein, for detecting or diagnosing a cancer, or treating, ameliorating or preventing a cancer, wherein optionally the cancer is:
a thyroid cancer, a lung cancer, or a carcinoma,
wherein optionally the carcinoma is a large cell carcinoma, a small cell carcinoma (optionally a small cell lung carcinoma or an extrapulmonary small cell carcinoma), a lung carcinoma or an adenocarcinoma, and optionally the adenocarcinoma is a pulmonary adenocarcinoma or a thyroid adenocarcinoma),
wherein optionally the cancer is a squamous carcinoma cell (SCC), a leukemia, a carcinoid tumor, a mamma carcinoma, a colon carcinoma cell, a malignant melanoma or a multiple myeloma, a plasmacytoma or a lymphoma or a cancer derived from a lymphocyte,

wherein optionally the lymphocyte is an early B cell, a pro-B cell, a pre-B lymphocyte, a mature B-lymphocyte, a T lymphocyte, a parafollicular T lymphocyte, a cell derived from a subpopulation of parafollicular T lymphocytes.

In alternative embodiments of uses as provided herein:
the Ab, or Ag binding fragment thereof, or monomeric or dimeric ABP is less sensitive (or binds with lower affinity) in specific binding to lung squamous cell carcinoma cells than specifically binding to adenocarcinoma cells, thereby resulting in fewer lung squamous cell carcinoma false positives and better positive identification of lung adenocarcinoma; and/or
the detection comprises conducting an immunohistochemistry (IHC) assay.

In alternative embodiments, provided are isolated or purified, or synthetic or recombinant antibodies (Ab), or antigen (Ag) binding fragments thereof, or monomeric or dimeric antigen binding proteins (ABP) as provided herein, for use in detecting or diagnosing a cancer, or treating, ameliorating or preventing a cancer; and optionally the cancer is:
a thyroid cancer, a lung cancer, or a carcinoma,
wherein optionally the carcinoma is a large cell carcinoma, a small cell carcinoma (optionally a small cell lung carcinoma or an extrapulmonary small cell carcinoma), a lung carcinoma or an adenocarcinoma, and optionally the adenocarcinoma is a pulmonary adenocarcinoma or a thyroid adenocarcinoma),
wherein optionally the cancer is a squamous carcinoma cell (SCC), a leukemia, a carcinoid tumor, a mamma carcinoma, a colon carcinoma cell, a malignant melanoma or a multiple myeloma, a plasmacytoma or a lymphoma or a cancer derived from a lymphocyte,

wherein optionally the lymphocyte is an early B cell, a pro-B cell, a pre-B lymphocyte, a mature B-lymphocyte, a T lymphocyte, a parafollicular T lymphocyte, a cell derived from a subpopulation of parafollicular T lymphocytes.

In alternative embodiments, of these isolated or purified, or synthetic or recombinant antibody (Ab), or antigen (Ag) binding fragment thereof, or monomeric or dimeric antigen binding protein (ABP) as provided herein: the Ab, or Ag binding fragment thereof, or monomeric or dimeric ABP is less sensitive (or binds with lower affinity) in specific binding to lung squamous cell carcinoma cells than specifically binding to adenocarcinoma cells, thereby resulting in fewer lung squamous cell carcinoma false positives and better positive identification of lung adenocarcinoma; or, the detecting or diagnosing comprises conducting an immunohistochemistry (IHC) assay.

In alternative embodiments, provided are kits comprising: a isolated or purified, or synthetic or chimeric or recombinant antibody as provided herein; a chimeric or a recombinant nucleic acid as provided herein; or an expression cassette, vector, recombinant virus, artificial chromosome, cosmid or plasmid as provided herein; or, a cell as provided herein; or a recombinant antibody (Ab), or antigen (Ag) binding fragment thereof, or monomeric or dimeric antigen binding protein (ABP) as provided herein. In alternative embodiments of kits as provided herein, the kit comprises components needed for an immunohistochemistry (IHC) assay, and/or comprises instructions for practicing a method as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 illustrates an IHC image of normal lung, showing specific staining of Type II cells (arrow), as discussed in detail in Example 1, below.

FIG. 2 illustrates an IHC image of a lung bronchi stained with clone 6F6 antibody, as discussed in detail in Example 1, below.

FIG. 3 illustrates an IHC image of a normal thyroid stained with clone 6F6 antibody, as discussed in detail in Example 1, below.

FIG. 4 illustrates an IHC image of negative normal tissue, a normal liver, showing hepatocytes (arrow), as discussed in detail in Example 1, below.

FIG. 5 illustrates an IHC image of lung adenocarcinoma, showing specific staining of the tumor cells, as discussed in detail in Example 1, below.

FIG. 6 illustrates an IHC image of lung squamous cell carcinoma, showing no specific staining of the tumor cells, as discussed in detail in Example 1, below.

FIG. 7 illustrates an IHC image of colon adenocarcinoma, showing no specific staining of the tumor cells, as discussed in detail in Example 1, below.

FIG. 8 graphically illustrates data showing specificity towards different tumor types: specificity was tested on a multi tumor array (MTU951) with antibody clones 6F6 and SPT24, as discussed in detail in Example 1, below.

FIG. 9 graphically illustrates data showing sensitivity towards lung adenocarcinoma, where specificity was tested on 22 lung adenocarcinomas with clones 6F6, SPT24, and 8G7G3/1, and results showed that clones 6F6 and SPT24 were more sensitive than clone 8G7G3/1, and that clone SPT24 was most sensitive, as discussed in detail in Example 1, below.

FIG. 10 graphically illustrates data showing specificity towards lung squamous cell carcinoma: specificity was tested on 32 lung squamous cell carcinomas with clones 6F6, SPT24, and 8G7G3/1, and results showed that clone SPT24 was the least specific clone, as discussed in detail in Example 1, below.

Like reference symbols in the various drawings indicate like elements.

### DETAILED DESCRIPTION

In alternative embodiments, provided are antibodies capable of specifically binding to lung adenocarcinoma cells, including providing chimeric, synthetic or recombinant anti-human homeobox NKX-2.1 (also called NK2 homeobox 1, or thyroid transcription factor 1 (TTF-1)) protein antibodies (Abs), including products of manufacture and kits comprising them, and methods for making and using them, including for example their use in the detection or diagnosis, and treatment, of a cancer, or other diseases or conditions involving expression of TTF-1. In alternative embodiments, anti-TTF-1 antibodies as provided herein are used in assays such as immunohistochemistry (IHC) assays, optionally together with a detecting or detectable agent, for determining whether TTF-1 expression or activity is present, reduced or absent on a cell, for example, detecting TTF-1 expression on a cell in a biopsy or other tissue or cell sample.

In alternative embodiments, an Ab, or antigen (Ag) binding fragment or protein (ABP) thereof, or monomeric or dimeric ABP, as provided herein is less sensitive (or binds with lower affinity) in specific binding to a lung squamous cell carcinoma cell than specifically binding to an adenocarcinoma cell, thereby resulting in fewer lung squamous cell carcinoma false positives and better positive identification of lung adenocarcinoma.

In alternative embodiments, clinical use of a TTF-1 assay, for example, a TTF-1 IHC assay, is to distinguish adenocarcinoma from squamous cell carcinoma in non-small cell lung cancer, as described for example by Yatabe et al (1). Yatabe et al discussed how the diagnostic sensitivity and specificity of anti-TTF-1 antibodies (Ab) produced by Agilent Dako clone 8G7G3/1 (IR056) differ from anti-TTF-1 antibodies produced by clone SPT24. This difference in sensitivity and specificity between the two anti-TTF-1 antibodies has been reported multiple times (2, 3). Provided herein is a new, improved anti-TTF-1 antibody produced by a new clone, where the new anti-TTF-1 antibody as provided herein has a diagnostic sensitivity comparable to the SPT24 anti-TTF-1 Ab and a diagnostic specificity comparable to the 8G7G3/1 anti-TTF-1 Ab.

### Expression of Recombinant Chimeric Antibodies

In alternative embodiments, chimeric or recombinant Abs as provided herein, including the exemplary chimeric or recombinant anti-human TTF-1 Ab, with signal peptide, or without the signal peptide, can be expressed as a recombinant Ab using a plasmid (or any expression vehicle) encoding the respective heavy and light chains, or the heavy chain and the light chain can be encoded in separate expression vehicles.

In some embodiments, the heavy and light chains can be (cis- or trans-) expressed from a pTT5™ vector(s) (National Research Council Canada, NRC-CNRC, Canada) in HEK293-6E cells. In alternative embodiment, the vector or vectors expressing the heavy and/or light chains are episomal or are chromosomally integrated, for example, in a stable cell line capable of synthesizing, optionally inducibly synthesizing, the heavy and/or light chains.

In alternative embodiments, provided are nucleic acids encoding chimeric or recombinant Abs as provided herein. Nucleic acids as provided herein can be made, isolated and/or manipulated by, for example, cloning and expression of cDNA libraries, amplification of message or genomic DNA by PCR, and the like. Nucleic acids used to practice embodiments as provided herein, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, fungal, mammalian, yeast, insect or plant cell expression systems.

Alternatively, these nucleic acids can be synthesized in vitro by well-known chemical synthesis techniques, as described in, for example, Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, for example, subcloning, labeling probes (for example, random-primer labeling using Klenow polymerase, nick translation, amplification), sequencing, hybridization and the like are well described in the scientific and patent literature, see, for example, Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice embodiments as provided herein comprises screening and re-cloning inserts isolated or amplified from, for example, genomic clones or cDNA clones. Sources of nucleic acids include recombinant nucleic acid sequences, genomic or cDNA libraries contained and/or expressed in, for example, mammalian artificial chromosomes (MACs), see, for example, U.S. Pat. Nos. 5,721,118; 6,025,155; human artificial chromosomes, see, for example, Rosenfeld (1997) Nat. Genet. 15:333-335; yeast artificial chromosomes (YAC); bacterial artificial chromosomes (BAC); P1 artificial chromosomes, see, for example, Woon (1998) Genomics 50:306-316; P1-derived vectors (PACs), see, for example, Kern (1997) Biotechniques 23:120-124; cosmids, recombinant viruses, phages, phagemids or plasmids.

In alternative embodiments, nucleic acids as provided herein are operably linked to transcriptional regulatory elements, including promoters, with can be constitutive or inducible transcriptional regulatory elements.

In alternative aspects, provided are “expression cassettes” comprising a nucleotide sequence as provided herein, for example encoding a chimeric or recombinant antibody as provided herein. Expression cassettes can include at least a transcriptional regulatory element, for example, a promoter, operably linked with an antibody coding sequence, and optionally can also include transcription termination signals. Additional factors necessary or helpful in effecting expression may also be used, for example, enhancers.

In alternative aspects, expression cassettes used to practice embodiments as provided herein include plasmids, expression vectors, recombinant viruses, any form of recombinant “naked DNA” vector, and the like. In alternative aspects, a “vector” used to practice embodiments as provided herein can comprise a nucleic acid that can infect, transfect, transiently or permanently transduce a cell. In alternative aspects, a vector used to practice embodiments as provided herein can be a naked nucleic acid, or a nucleic acid complexed with protein or lipid. In alternative aspects, vectors used to practice embodiments as provided herein can comprise viral or bacterial nucleic acids and/or proteins, and/or membranes (for example, a cell membrane, a viral lipid envelope, etc.). In alternative aspects, vectors used to practice embodiments as provided herein can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, the vector used to practice embodiments as provided herein can be stably replicated by the cells during mitosis as an autonomous structure, or can be incorporated within the host's genome.

In alternative aspects, “promoters” used to practice embodiments as provided herein include all sequences capable of driving transcription of a coding sequence in a cell, for example, a bacterial, yeast, fungal, plant, insect (for example, baculovirus) or mammalian cell. Thus, promoters used in the constructs include cis-acting transcriptional control elements and regulatory sequences that are involved in regulating or modulating the timing and/or rate of transcription of a gene. For example, a promoter used to practice embodiments as provided herein can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription.

“Constitutive” promoters used to practice embodiments as provided herein can be those that drive expression continuously under most environmental conditions and states of development or cell differentiation. “Inducible” or “regulatable” promoters used to practice embodiments as provided herein can direct expression of a nucleic acid as provided herein under the influence of environmental conditions or developmental conditions. Examples of environmental conditions that may affect transcription by inducible promoters used to practice embodiments as provided herein include the presence of an inducing factor administered to a cell.

In alternative embodiments, peptides and polypeptides used to practice embodiments as provided herein can comprise any “mimetic” and/or “peptidomimetic” form. In alternative embodiments, peptides and polypeptides used to practice embodiments as provided herein can comprise synthetic chemical compounds which have substantially the same structural and/or functional characteristics of the natural polypeptide, for example, a chimeric or recombinant antibody as provided herein. The mimetic used to practice embodiments as provided herein can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Routine experimentation will determine whether a mimetic is effective for practicing the invention, for example, if a mimetic composition is effective in specifically binding TTF-1 protein. Methodologies detailed herein and others known to persons skilled in the art may be used to select or guide one to choose effective mimetic for practicing the compositions and/or methods of this invention.

Polypeptide mimetic compositions for practicing embodiments as provided herein can comprise any combination of non-natural structural components. In alternative aspects, mimetic compositions for practicing embodiments as provided herein can comprise one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, for example, a beta turn, gamma turn, beta sheet, alpha helix conformation, and the like. For example, a polypeptide can be characterized as a mimetic when all or some of its residues are joined by chemical means other than natural peptide bonds.

### Summary of Amino Acid Sequences of Polypeptides and Peptides as Provided Herein:

Heavy chain variable domain
(SEQ ID NO: 1)
QSLKESEGGLFKPTDTLTLTCTVSGFSLSSYAMIWVRQAPGNGLEYIGA
INSYGNAYYASWAKSRSTITRDTNENTVTLKMTSLTAADTATYFCTTTH
SYGFGAFDPWGPGTLVTVSS
Light chain variable domain
(SEQ ID NO: 2)
AAVLTQTPSPVSAAVGGTVSISCQSSESAYNNRLAWYQQKPGQPPKLLI
YDASKLASGVPSRFKGSGSGTPFTLTISGVQCADAATYYCQGVYSSVSD
VYAFGGGTEVVVK
VH Constant
(SEQ ID NO: 3)
GQPKAPSVFPLAPCCGDTPSSTVTLGCLVKGYLPEPVTVTWNSGTLTNG
VRTFPSVRQSSGLYSLSSVVSVTSSSQPVTCNVAHPATNTKVDKTVAPS
TCSKPTCPPPELLGGPSVFIFPPKPKDTLMISRTPEVTCVVVDVSQDDP
EVQFTWYINNEQVRTARPPLREQQFNSTIRVVSTLPIAHQDWLRGKEFK
CKVHNKALPAPIEKTISKARGQPLEPKVYTMGPPREELSSRSVSLTCMI
NGFYPSDISVEWEKNGKAEDNYKTTPAVLDSDGSYFLYSKLSVPTSEWQ
RGDVFTCSVMHEALHNHYTQKSISRSPGK
Full H chain
(SEQ ID NO: 4)
QSLKESEGGLFKPTDTLTLTCTVSGFSLSSYAMIWVRQAPGNGLEYIGA
INSYGNAYYASWAKSRSTITRDTNENTVTLKMTSLTAADTATYFCTTTH
SYGFGAFDPWGPGTLVTVSSGQPKAPSVFPLAPCCGDTPSSTVTLGCLV
KGYLPEPVTVTWNSGTLTNGVRTFPSVRQSSGLYSLSSVVSVTSSSQPV
TCNVAHPATNTKVDKTVAPSTCSKPTCPPPELLGGPSVFIFPPKPKDTL
MISRTPEVTCVVVDVSQDDPEVQFTWYINNEQVRTARPPLREQQFNSTI
RVVSTLPIAHQDWLRGKEFKCKVHNKALPAPIEKTISKARGQPLEPKVY
TMGPPREELSSRSVSLTCMINGFYPSDISVEWEKNGKAEDNYKTTPAVL
DSDGSYFLYSKLSVPTSEWQRGDVFTCSVMHEALHNHYTQKSISRSPGK
VH SignalP
(SEQ ID NO: 5)
METGLRWLLLVAVLKGVQC
VH + SignalP
(SEQ ID NO: 6)
METGLRWLLLVAVLKGVQCQSLKESEGGLFKPTDTLTLTCTVSGFSLSS
YAMIWVRQAPGNGLEYIGAINSYGNAYYASWAKSRSTITRDTNENTVTL
KMTSLTAADTATYFCTTTHSYGFGAFDPWGPGTLVTVSS
Full H chain + SignalP
(SEQ ID NO: 7)
METGLRWLLLVAVLKGVQCQSLKESEGGLFKPTDTLTLTCTVSGFSLSS
YAMIWVRQAPGNGLEYIGAINSYGNAYYASWAKSRSTITRDTNENTVTL
KMTSLTAADTATYFCTTTHSYGFGAFDPWGPGTLVTVSSGQPKAPSVFP
LAPCCGDTPSSTVTLGCLVKGYLPEPVTVTWNSGTLTNGVRTFPSVRQS
SGLYSLSSVVSVTSSSQPVTCNVAHPATNTKVDKTVAPSTCSKPTCPPP
ELLGGPSVFIFPPKPKDTLMISRTPEVTCVVVDVSQDDPEVQFTWYINN
EQVRTARPPLREQQFNSTIRVVSTLPIAHQDWLRGKEFKCKVHNKALPA
PIEKTISKARGQPLEPKVYTMGPPREELSSRSVSLTCMINGFYPSDISV
EWEKNGKAEDNYKTTPAVLDSDGSYFLYSKLSVPTSEWQRGDVFTCSVM
HEALHNHYTQKSISRSPGK
VL constant
(SEQ ID NO: 8)
GDPVAPTVLIFPPAADQVATGTVTIVCVANKYFPDVTVTWEVDGTTQTT
GIENSKTPQNSADCTYNLSSTLTLTSTQYNSHKEYTCKVTQGTTSVVQS
FNRGDC
Full L chain
(SEQ ID NO: 9)
AAVLTQTPSPVSAAVGGTVSISCQSSESAYNNRLAWYQQKPGQPPKLLI
YDASKLASGVPSRFKGSGSGTPFTLTISGVQCADAATYYCQGVYSSVSD
VYAFGGGTEVVVKGDPVAPTVLIFPPAADQVATGTVTIVCVANKYFPDV
TVTWEVDGTTQTTGIENSKTPQNSADCTYNLSSTLTLTSTQYNSHKEYT
CKVTQGTTSVVQSFNRGDC
VL + SignalP
(SEQ ID NO: 10)
MDTRAPTQLLGLLLLWLPGATFAAVLTQTPSPVSAAVGGTVSISCQSSE
SAYNNRLAWYQQKPGQPPKLLIYDASKLASGVPSRFKGSGSGTPFTLTI
SGVQCADAATYYCQGVYSSVSDVYAFGGGTEVVVK
Full L chain + SignalP
(SEQ ID NO: 11)
MDTRAPTQLLGLLLLWLPGATFAAVLTQTPSPVSAAVGGTVSISCQSSE
SAYNNRLAWYQQKPGQPPKLLIYDASKLASGVPSRFKGSGSGTPFTLTI
SGVQCADAATYYCQGVYSSVSDVYAFGGGTEVVVKGDPVAPTVLIFPPA
ADQVATGTVTIVCVANKYFPDVTVTWEVDGTTQTTGIENSKTPQNSADC
TYNLSSTLTLTSTQYNSHKEYTCKVTQGTTSVVQSFNRGDC
VL SignalP
(SEQ ID NO: 12)
MDTRAPTQLLGLLLLWLPGATF

### Immunohistochemistry (IHC)

In alternative embodiments, immunohistochemistry (IHC) methodologies and/or reagents used to practice compositions, products of manufacture, kits or methods as provided herein can include or comprise or comprise use of any IHC protocol, IHC armamentarium, device and/or image or data analysis system, for practicing IHC or IHC reagents known in the art, for example, as described in U.S. Pat. No. 11,321,881 (describing IHC imaging protocols and apparatus); U.S. Pat. No. 11,249,085, describing applications of click chemistry for signal amplification in IHC and ISH assays; U.S. Pat. No. 11,222,424 (describing IHC imaging apparatus and computer programs); U.S. Pat. No. 11,143,648, describing new colors for chromogenic IHC and ISH staining with multi-dye quinone methide and tyramide conjugates; U.S. Pat. No. 11,047,774 (describing automated IHC specimen processing systems); U.S. Pat. No. 11,028,044, describing processes for commercial scale preparation of 3,3′5,5′-tetramethylbenzidine (TMB) and its salts; U.S. Pat. No. 10,977,791, describing computer-implemented methods for analysis of a tissue sample; U.S. Pat. No. 10,816,443, describing automated batch stainers for staining biological specimens on microscope slides; U.S. Pat. No. 10,718,773, describing methods for determining the eligibility of a subject having a malignancy for treatment with a therapeutic agent; U.S. Pat. No. 10,565,479 (describing methods for identifying blurred areas in digital images of stained tissue); U.S. Pat. No. 10,564,076 (describing systems for analytical (or IHC) sample preparation); U.S. Pat. No. 10,551,395 (describing an automated histological staining system); U.S. Pat. No. 10,551,378 (describing a tissue staining method); U.S. Pat. No. 10,504,224 (describing a digital tissue image analysis system for IHC); U.S. Pat. No. 10,501,777 (describing simultaneous, multiplexed detection and quantification of protein expression in IHC); U.S. Pat. No. 10,488,340 (describing method for extracting an image of a target fluorophore in a biological material); U.S. Pat. No. 10,453,195 (describing methods of detecting tissue areas of interest using digital pathology imaging); U.S. Pat. No. 10,438,381 (describing devices, systems and methods for generating a digital image of a tissue section); U.S. Pat. No. 10,416,176 (describing methods for processing specimens in an automated histological staining system); U.S. Pat. No. 10,393,633 (describing methods for processing and inhibiting the degradation of an IHC sample); U.S. Pat. No. 10,217,011 (describing handling of IHC slides); U.S. Pat. No. 10,209,165 (describing automated or semi-automated methods for assessing the quality of staining of a specimen containing cells); U.S. Pat. No. 10,126,216 (describing methods for fixing tissue samples for IHC); 9,423,322; or as described in U.S. patent application publication nos. US 2021/0239683A1, describing compositions for forming a porous hydrogel around a cell suitable for immunostaining of cells within the hydrogel; or U.S. Pat. No. 11,112,413, or US 2022/0057408 A1, describing methods of employing the epitope-tagged antibodies for detecting one or more targets in an IHC tissue sample; or US 2021/0048432 A1, describing direct immunohistochemical (IHC) staining and direct immunocytochemistry (ICC) techniques; or US 2020/0292536, describing synthetic controls useful in immunohistochemistry (IHC) assays; or US 20220057408, describing use of antibodies with epitope tags in immunohistochemistry (IHC) assays; or US 20210201485, describing computer-implemented methods for analysis of a tissue sample; US 2019/0178867 A1 (describing detection of specific tissue objects within thin sections of tissue samples as imaged in a brightfield microscope without using a chromogenic stain that is specific to those tissue objects); US 2019/0156510 A1 (describing an image analysis method for analyzing an IHC tissue sample); US 2019/0293637 A1 (methods and systems for quantitative immunohistochemistry (IHC) of a target protein molecule); US 2019/0080450 A1 (describing an automated determination of the staining quality of an IHC stained biological sample); or, US 2020/0316589 A1 (describing a multi-well solid support vessel for the processing and testing of fixed biological materials).

In alternative embodiments, chimeric or the recombinant antibodies, antigen binding fragments thereof, or monomeric or dimeric antigen binding proteins, in IHC protocols, or kits, as provided herein are substantially purified or isolated or are in the form of an unpurified or partially purified culture supernatant.

In alternative embodiments, methods as provided herein can use or comprise reagents for detecting or visualizing an antibody-antigen interaction using any products or methods know in the art, for example, and IHC protocol or reagents.

In alternative embodiments, methods as provided herein comprise use of chromogenic immunohistochemistry (CIH), wherein a primary antibody (for example, chimeric or a recombinant antibodies (Ab), or antigen binding fragments thereof, or monomeric or dimeric antigen binding proteins as provided herein) or secondary antibody (for example, where the secondary antibody binds to (the primary antibody) chimeric or a recombinant antibodies (Ab), or antigen binding fragments thereof, or monomeric or dimeric antigen binding proteins as provided herein after they have specifically bound to, paired with or associated with, a TTF-1 epitope or polypeptide) is conjugated to an enzyme, such as peroxidase (or immunoperoxidase), for example, a horseradish peroxidase (HRP), that can catalyze a color-producing reaction.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors. 3,3′-Diaminobenzidine (DAB) also can be used.

In alternative embodiments, methods as provided herein comprise use of a direct method or one-step staining method where a primary antibody (for example, chimeric or a recombinant antibodies (Ab), or antigen binding fragments thereof, or monomeric or dimeric antigen binding proteins as provided herein) is labeled and reacts directly with an antigen, for example, in a tissue sections. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (for example, TTF-1), for example, in a tissue or organ, and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules, for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or formalin-fixed paraffin-embedded tissues. In alternative embodiments, a tissue is sliced or used whole. Before sectioning, the tissue sample can be embedded in a medium, for example, paraffin wax or cryomedia. Tissue sections can be sliced on a variety of instruments, most commonly using a microtome, cryostat, or vibratome. Specimens can be sliced at a range of about 3 μm to 5 μm. The slices can be mounted on slides, dehydrated using alcohol washes of increasing concentrations (for example, 50%, 75%, 90%, 95%, 100%), and cleared using a detergent like xylene before being imaged under a microscope.

Depending on the method of fixation and tissue preservation, the sample may require additional steps to make the TTF-1 epitopes available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (EnVision DuoFLEX Doublestain System) (Agilent, San Jose, CA), which allows for staining of two or more markers on a single slide. In alternative embodiments, the IHC is performed using an EnVision FLEX HRP Magenta, High pH (Dako OMNIS™) system, and binding can be visualized by EnVision FLEX HRP™ Magenta Chromogen. In alternative embodiments, the IHC is performed using EnVision FLEX Mini Kit™, High pH, which is a high-sensitivity visualization system intended for use in IHC together with Dako AUTOSTAINER™ instruments: this dual link system detects primary mouse and rabbit antibodies and the reaction is visualized by 3,3′-Diaminobenzidine (DAB) chromogen (DAB forms a water-insoluble brown precipitate when oxidized, for example, by a peroxidase).

### Products of Manufacture and Kits

Provided are products of manufacture and kits for practicing methods as provided herein, for example, comprising chimeric or recombinant anti-TTF-1 polypeptide Abs as provided herein; and optionally the products of manufacture and kits can further comprise some or all reagents needed to perform an IHC, and optionally can comprise instructions for practicing methods as provided herein.

In alternative embodiments, the products of manufacture, or kits, comprise mixtures or cocktails of antibodies (Abs) as provided herein, for example, a mixture or cocktail comprising two, three or more anti-human TTF-1 Ab antibodies.

In alternative embodiments, the products of manufacture, or kits, comprise mixtures or cocktails of antibodies (Abs) comprising antibodies comprising heavy chain and/or light chain CDRs of antibodies as provided herein, or as produced by antibody-producing clones as provided herein.

In alternative embodiments, the products of manufacture, or kits, comprise antibody-producing clones as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Antibodies for Identifying and Distinguishing Lung Adenocarcinoma from Lung Squamous Cell Carcinoma

In alternative embodiments, an Ab, or antigen (Ag) binding fragment or protein (ABP) thereof, or monomeric or dimeric ABP, as provided herein is less sensitive (or binds with lower affinity) in specific binding to a lung squamous cell carcinoma cell than specifically binding to an adenocarcinoma cell, thereby resulting in fewer false positives in binding to lung squamous cell carcinoma cells and better identification of lung adenocarcinoma.

Yatabe et al (1) show that the antibody of clone 8G7G3/1 (producing an anti-hunan TTF-1 antibody) is more specific in binding to TTF-1 than the anti-human TTF-1 antibody produced by clone SPT24, but the anti-human TTF-1 antibody produced by clone SPT24 is more sensitive.

Described here is a new anti-human TTF-1 antibody TTF-1 assay which is as specific as the anti-human TTF-1 antibody produced by clone 8G7G3/1 and as sensitive as the anti-human TTF-1 antibody produced by clone SPT24. The goal is to have as many positive lung adenocarcinomas as identified using the anti-human TTF-1 antibody of SPT24 and to have as few positive squamous cell carcinomas as seen with the anti-human TTF-1 antibody of clone 8G7G3/1.

Because a lot of samples were needed to be tested with the three different assays, including clone SPT24, clone 8G7G3/1 and the new clone 6F6 as described herein, antibody sensitivity and specificity was tested on a tissue microarray (TMA), specifically, a lung cancer mid-density tissue array, LUC1501™, and a multi-tumor array MTU951™, from US Biomax (Derwood, MD).

The anti-human TTF-1 antibody of clone 8G7G3/1 is less sensitive than the anti-human TTF-1 antibody of clone SPT24 on lung adenocarcinomas (false negatives), but more specific on lung squamous cell carcinoma (fewer false positives). See for example the Table below excerpted from Yatabe et al (1).

| | 8G7G3/1 | | SPT24 | |
|---|---|---|---|---|
| Carcinoma | n | Positive, n (%) | n | Positive, n (%) |

TTF-1 expression in lung adenocarcinoma and squamous cell carcinoma

| | | | | | | |
|---|---|---|---|---|---|---|
| Lung | 2614 | 2004 | (76.7%) | 579 | 471 | (81.3%) |
| adenocarcinoma | | | | | | |

Squamous cell carcinoma

| | | | | | | |
|---|---|---|---|---|---|---|
| Kadota et al. | 449 | 0 | (0%) | 448 | 27 | (6.0%) |

| | | | | |
|---|---|---|---|---|
| Kashima et al. | 38 | 1 (3%) with | 38 | 5 (13%) with |
| | | En Vision | | EnVision |
| | 38 | 4 (11%) with | 38 | 20 (53%) with |
| | | CSA-II | | CSA-II |

The expected expression pattern in both normal and neoplastic tissue is outlined below.
Normal Lung: The type II pneumocytes, the Clara cells (or nonciliated bronchiolar secretory cells) and the basal cells of the terminal bronchioles shall show a strong nuclear staining reaction without significant cytoplasmic reaction. Depending on the sensitivity of the assay the columnar epithelial cells of the terminal bronchioles wall may show a moderate to strong nuclear staining reaction.
In normal thyroid the follicular epithelial cells shall show a strong nuclear staining reaction.
In normal Liver the hepatocytes will be negative.
Neoplastic Lung: In lung adenocarcinoma the tumor cells will show a moderate to strong nuclear staining reaction. In lung squamous cell carcinoma the tumor cells shall be negative.

### Clone Selection

Rabbits were immunized with recombinant protein expressed in E. coli. representing amino acids 1-156 of human TTF-1. Rabbit bleeds were evaluated by ELISA and IHC and candidate rabbits were chosen for B-cell selections. Several candidate clones were identified; IHC and cloning was performed of one B-cell clone: clone 6F6, yielding functional heavy and light chain sequences. Recombinant antibody was produced in HEK cells of clone 6F6 and tested in IHC, showing specificity towards TTF-1. The new rabbit anti-human TTF-1 antibody, clone 6F6, was tested on a variety of normal and neoplastic tissues to ascertain specificity and sensitivity and was compared to clone SPT24 and 8G7G3/1 for specificity and sensitivity.

The tissues included:

### Neoplastic Tissue

Lung adenocarcinoma (clinically relevant positive tissue)
Lung squamous cell carcinoma (clinically relevant negative tissue)
Colon adenocarcinoma (shall be negative)
Lung carcinoid (may be positive)
Small-cell carcinoma (may be positive)
Multi tumor array (MTU951)

### Normal Tissue

Large multi block containing 12 different normal and neoplastic
   30 different normal tissues tissues
Large multi block containing 12 different normal and neoplastic tissues

### Reaction Pattern New Assay

Weaker on the low expressing epithelial cell of bronchi than clone SPT24. The rationale being that with a more sensitive protocol, clone 6F6 would have the same issues as clone SPT24 with a risk of increased false positive staining of squamous cell carcinomas.

Background staining of mucus producing cells of stomach. This staining was also observed, although to a lesser degree, with the reference clone SPT24. While unwanted the reaction does not pose any diagnostic risk or risk for being unable to interpret specific staining.

No reaction was observed in normal hepatocytes. Clone 8G7G3/1 does indeed stain hepatocytes to a high degree.

Some positivity on colon adenocarcinoma: Some colon adenocarcinomas were weakly positive in the same areas as were positive with the SPT24 clone. Hence, the new clone does not seem to be more specific in that regard, but instead the new assay has lower staining intensity.

Multi-tumor array, MTU951, showed specific staining in the tissues where it was expected. Clone SPT24 has unexpected staining of one skin squamous cell carcinoma.

Lung tumor tissue micro-array LUC1501, showed lower intensity staining with the new assay compared to the reference assay using SPT24. However, there were no false negative cases (although weaker staining). The difference in intensity was typically within the range of 0.5 to 2. One clinical tissue had higher staining intensity with clone 6F6 than SPT24, on all other tissues the staining intensity was weaker. Nine out of 32 negative clinical tissues were positive with clone SPT24, while 5 out of the 32 were positive with clone 6F6

The new clone 6F6 was tested on 30 normal tissues in triplicate (different cases). The reaction was as expected, except staining of mucus producing cells in the stomach.

| | # positive/ | | # positive/ |
|---|---|---|---|
| Tissue | total cases | Tissue | total cases |
| Peripheral Nerve | 0/3 | Parathyroid | 3/3 |
| Adrenal | 0/3 | Pituitary | 0/3 |
| Bone marrow | 0/3 | Prostate | 0/3 |
| Cerebellum | 0/3 | Salivary Gland | 0/3 |
| Cerebrum | 0/3 | Skeletal muscle | 0/3 |
| Cervix | 0/3 | Skin | 0/3 |
| Colon | 0/3 | Small intestine | 0/3 |
| Esophagus | 0/3 | Spleen | 0/3 |
| Heart | 0/3 | Stomach* | 0/3 |
| Kidney | 0/3 | Testis | 0/3 |
| Liver | 0/3 | Thymus | 0/3 |
| Lung | 3/3 | Thyroid | 3/3 |
| Mesothelial cells | 0/3 | Tonsil | 0/3 |
| Ovary | 0/3 | Uterus | 0/3 |
| Pancreas | 0/3 | Breast | 0/3 |

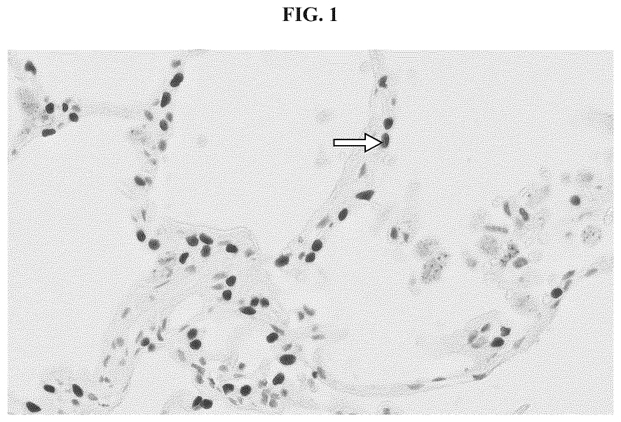

FIG. 1 illustrates an IHC image of normal lung, showing specific staining of Type II cells (arrow). Stained with clone 6F6 antibody.

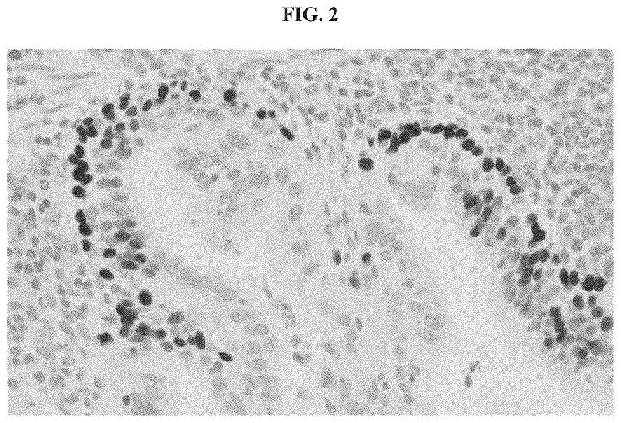

FIG. 2 illustrates an IHC image of a lung bronchi stained with clone 6F6 antibody.

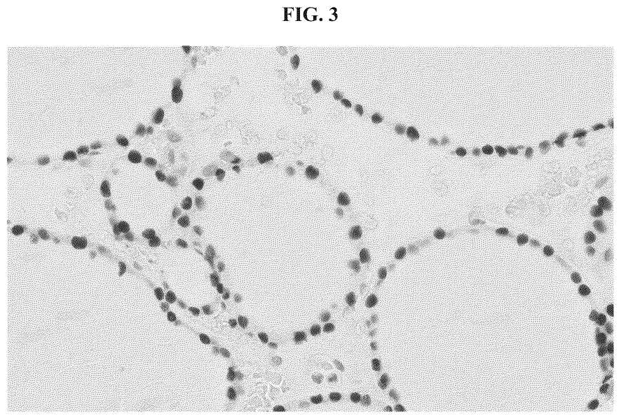

FIG. 3 illustrates an IHC image of a normal thyroid stained with clone 6F6 antibody.

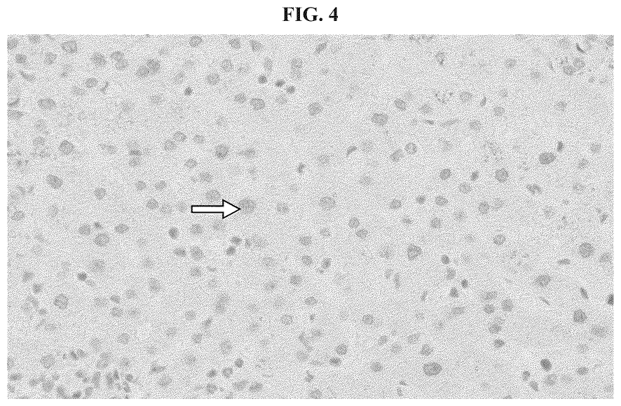

FIG. 4 illustrates an IHC image of negative normal tissue, a normal liver, showing hepatocytes (arrow). Stained with clone 6F6 antibody.

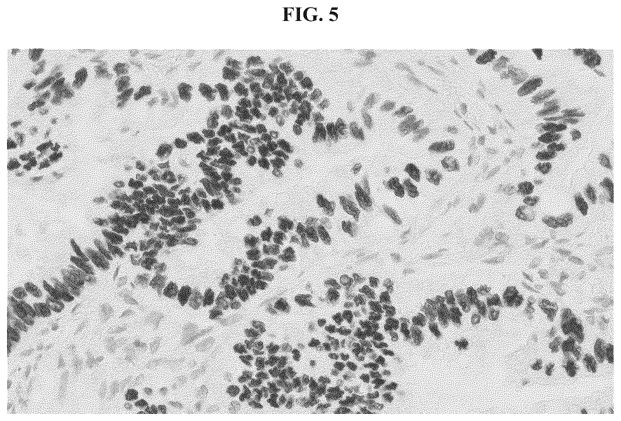

FIG. 5 illustrates an IHC image of lung adenocarcinoma, showing specific staining of the tumor cells. Stained with clone 6F6 antibody.

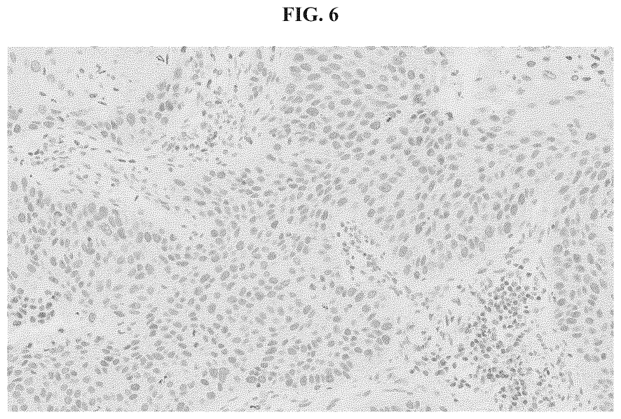

FIG. 6 illustrates an IHC image of lung squamous cell carcinoma, showing no specific staining of the tumor cells. Stained with clone 6F6 antibody.

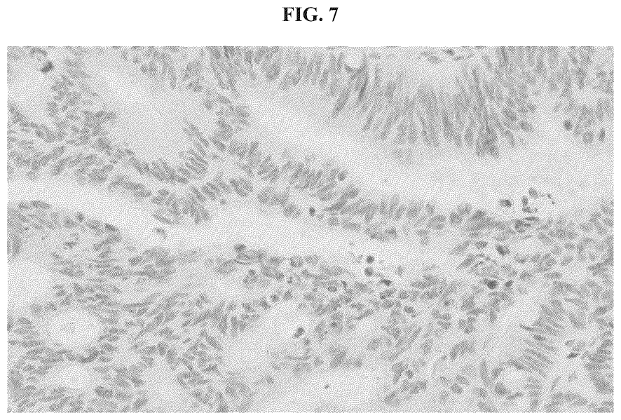

FIG. 7 illustrates an IHC image of colon adenocarcinoma, showing no specific staining of the tumor cells. Stained with clone 6F6 antibody.

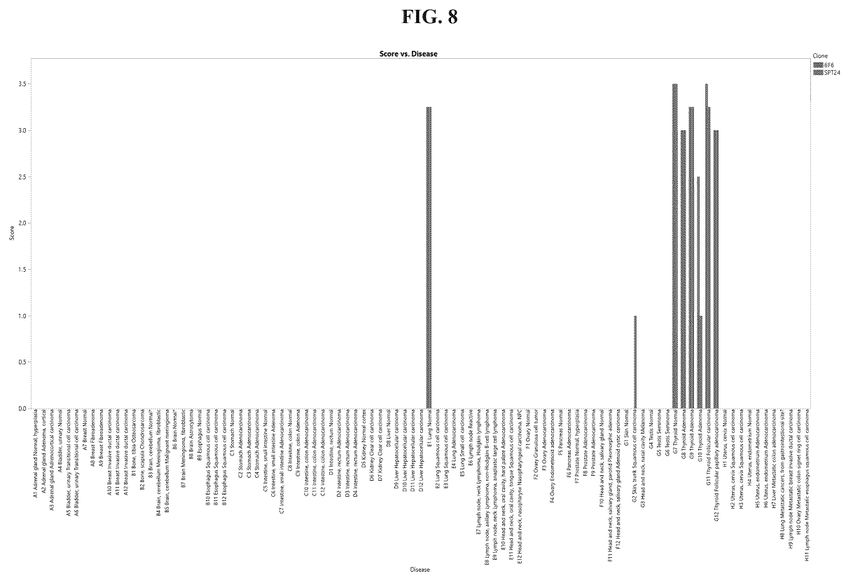

FIG. 8 illustrates: Specificity towards different tumor types: Specificity was tested on a multi tumor array (MTU951) with antibody clones 6F6 and SPT24. Positivity was as expected for clone 6F6, while clone SPT24 showed unspecific positive staining in one skin squamous cell carcinoma.

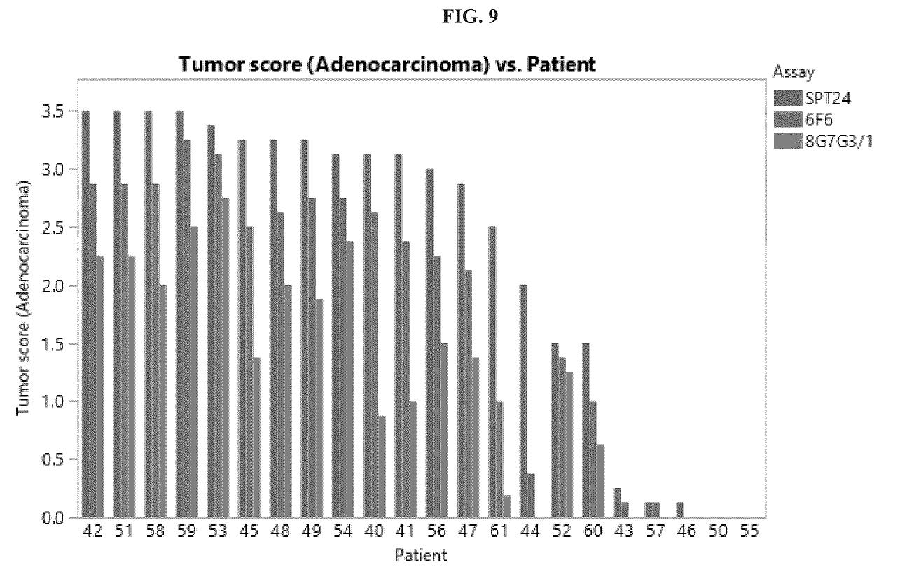

FIG. 9 illustrates: Sensitivity towards lung adenocarcinoma: Specificity was tested on 22 lung adenocarcinomas with clones 6F6, SPT24, and 8G7G3/1. Results showed that clones 6F6 and SPT24 were more sensitive than clone 8G7G3/1, and that clone SPT24 was most sensitive. Clone 8G7G3/1 was the least sensitive clone.

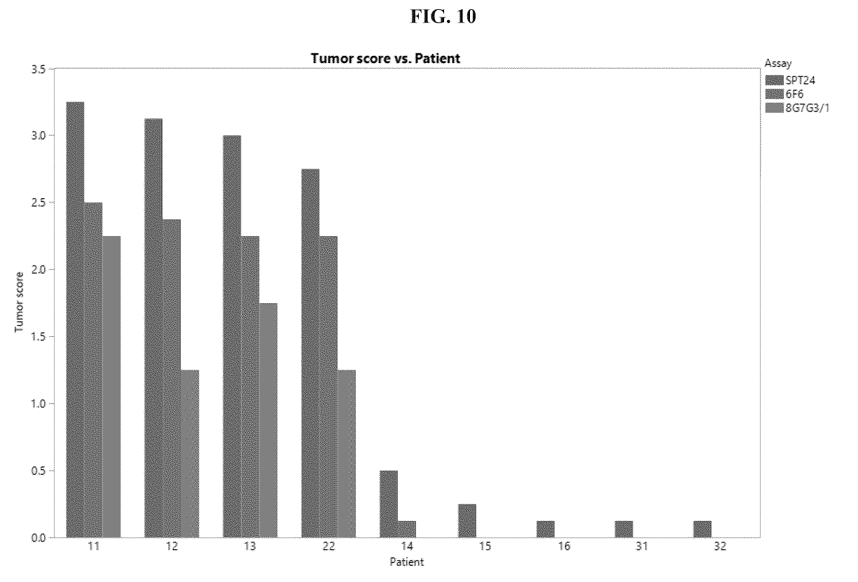

FIG. 10 illustrates: Specificity towards lung squamous cell carcinoma: Specificity was tested on 32 lung squamous cell carcinomas with clones 6F6, SPT24, and 8G7G3/1. Results showed that clone SPT24 was the least specific clone (positive in 9 of the 32 cases). Clone 8G7G3/1 was the most specific clone (positive in 4 of the 32 cases) followed by clone 6F6 (positive in 5 of the 32 cases).

### REFERENCES

1. Yatabe Y, et al. Best Practices Recommendations for Diagnostic Immunohistochemistry in Lung Cancer. J Thorac Oncol. 2019; 14(3):377-407.
2. Comperat E, et al. Variable sensitivity and specificity of TTF-1 antibodies in lung metastatic adenocarcinoma of colorectal origin. Mod Pathol. 2005; 18(10):1371-6.
3. Matoso A, et al. Comparison of thyroid transcription factor-1 expression by 2 monoclonal antibodies in pulmonary and nonpulmonary primary tumors. Appl Immunohistochem Mol Morphol. 2010; 18(2):142-9.
4. Holzinger A, et al. Monoclonal antibody to thyroid transcription factor-1: production, characterization, and usefulness in tumor diagnosis. Hybridoma. 1996; 15(1):49-53.
5. Lazzaro D, et al., The transcription factor TTF-1 is expressed at the onset of thyroid and lung morphogenesis and in restricted regions of the foetal brain. Development. 1991; 113(4):1093-104.

A number of embodiments of the invention have been described. Nevertheless, it can be understood that various modifications may be made without departing from the spirit and scope of the invention. Embodiments of the invention are set forth in the following claims.

1. An isolated or purified antibody (Ab), or synthetic or recombinant Ab, or antigen (Ag) binding fragment thereof, or monomeric or dimeric antigen binding protein (ABP), capable of specifically binding a human homeobox NKX-2.1 (also called NK2 homeobox 1, or thyroid transcription factor 1 (TTF-1)) protein or polypeptide,
wherein the isolated or purified Ab, or synthetic or recombinant Ab, or Ag binding fragment thereof, or monomeric or dimeric ABP comprises:
(a) a heavy chain variable region (VH) capable of specifically binding to a human homeobox NKX-2.1 protein or polypeptide, comprising:
(1) an amino acid sequence comprising the three CDR1, CDR2 and CDR3 complementarity determining regions (CDRs) of SEQ ID NO:1, or CDR1 amino acid (aa) residues GFSLSSYA (residues 25 to 32 of SEQ ID NO:1), CDR2 aa residues INSYGNA (residues 50 to 56 of SEQ ID NO:1), and CDR3 aa residues TTTHSYGFGAFDP (residues 95 to 107 of SEQ ID NO:1), or
(2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:1, or CDR1 amino acid (aa) residues GFSLSSYA (residues 25 to 32 of SEQ ID NO:1), CDR2 aa residues INSYGNA (residues 50 to 56 of SEQ ID NO:1), and CDR3 aa residues TTTHSYGFGAFDP (residues 95 to 107 of SEQ ID NO:1), or
(3) an amino acid sequence having at least about 70%, 75%, 80%, 85%, 90%, 95%, 98% sequence identity, or between about 70% to 100% sequence identity, to SEQ ID NO:1, or an amino acid sequence having complete sequence identity to SEQ ID NO:1;
(b) a light chain variable region (VL) capable of specifically bind to a human homeobox NKX-2.1 protein or polypeptide comprising:
(1) an amino acid sequence comprising the three CDR1, CDR2 and CDR3 complementarity determining regions (CDRs) of SEQ ID NO:2, or CDR1 amino acid (aa) residues ESAYNNR (residues 27 to 33 of SEQ ID NO:2), CDR2 aa residues DAS (residues 51 to 53 of SEQ ID NO:2), and CDR3 aa residues QGVYSSVSDVYA (residues 90 to 101 of SEQ ID NO:2), or
(2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:2, or CDR1 amino acid (aa) residues ESAYNNR (residues 27 to 33 of SEQ ID NO:2), CDR2 aa residues DAS (residues 51 to 53 of SEQ ID NO:2), and CDR3 aa residues QGVYSSVSDVYA (residues 90 to 101 of SEQ ID NO:2); or
(3) an amino acid sequence having at least about 70%, 75%, 80%, 85%, 90%, 95%, 98% sequence identity, or between about 70% to 100% sequence identity, to SEQ ID NO:2, or an amino acid sequence having complete (100%) sequence identity to SEQ ID NO:2; or
(c) a heterodimer capable of specifically bind to a human homeobox NKX-2.1 protein or polypeptide comprising the heavy chain variable region (VH) of (a) and the light chain variable region (VL) of (b).

2. The isolated or purified Ab, or synthetic or recombinant Ab, or Ag binding fragment thereof, or monomeric or dimeric ABP, of claim 1, fabricated as or in the form of:
an antigen-binding fragment (Fab, or an Ab fragment having just one constant and one variable domain of each of an Ab heavy and light chain),
a F(ab′)₂ (or an Ab digested by pepsin yielding two fragments: a F(ab′)₂ fragment and a pFc′ (pepsin cleavage Fc) fragment),
a Fab′ (a single chain of a F(ab′)₂ fragment),
a single-chain variable fragment (scFv) (or a fusion protein of a variable region of an Ab heavy and light chain connected together with a linker peptide optionally of about ten to about 25 amino acids in length),
a (scFv)₂, or a di-scFv or a bi-scFv, or a single peptide chain having two variable heavy and two variable light regions yielding tandem scFv,
a minibody (or a fusion protein of a variable region of an Ab heavy and light chain connected together with an alkyl group, optionally a methyl or an ethyl group)
a diabody (or an scFv with a linker peptide too short (optionally about five amino acids) for the two variable regions to fold together forcing the scFvs to dimerize), a triabody or a tetrabody (or an scFv with a linker peptide too short (optionally about one or two amino acids) for the two variable regions to fold together forcing the scFvs to trimerize or tetramize),
a single-domain antibody (dAB) (or a single variable region of an Ab heavy or Ab light chain),
a plurality of complementarity determining region (CDR) fragments, or
a multispecific antibody formed from two or more antibody fragments.

3. The isolated or purified Ab, or synthetic or recombinant Ab, or Ag binding fragment thereof, or monomeric or dimeric ABP, of claim 1, wherein the heavy chain variable region (VH), if present:
(a) further comprises a carboxy terminal constant domain, or further comprises amino acid sequence SEQ ID NO:3, or comprises SEQ ID NO:4;
(b) further comprises an amino terminal signal peptide, or further comprises an amino terminal signal peptide comprising: METGLRWLLLVAVLKGVQC (SEQ ID NO:5), or comprises SEQ ID NO:6 or SEQ ID NO:7;
(c) comprises SEQ ID NO:4, SEQ ID NO:6 or SEQ ID NO:7, having one or more amino acid substitutions, additions (insertions) or deletions, and the recombinant Ab, or Ag binding fragment thereof, or monomeric or dimeric ABP retains its ability to specifically bind to a human homeobox NKX-2.1 protein or polypeptide; or
(d) the one or more amino acid substitutions comprise one or more conservative amino acid substitutions.

4. The isolated or purified Ab, or synthetic or recombinant Ab, or Ag binding fragment thereof, or monomeric or dimeric ABP, of claim 1, wherein the light chain variable region (VL), if present:
(a) further comprises a carboxy terminal constant domain, or further comprises amino acid sequence SEQ ID NO:8, or comprises SEQ ID NO:9,
(b) further comprises an amino terminal signal peptide, or further comprises an amino terminal signal peptide consisting of MDTRAPTQLLGLLLLWLPGATF (SEQ ID NO:12), or comprises SEQ ID NO:10 or SEQ ID NO:11;
(c) comprises SEQ ID NO:9, SEQ ID NO:10 or SEQ ID NO:11, having one or more amino acid substitutions, additions (insertions) or deletions, and the recombinant Ab, or Ag binding fragment thereof, or monomeric or dimeric ABP retains its ability to specifically bind to a human homeobox NKX-2.1 protein or polypeptide,
(d) the one or more amino acid substitutions comprise one or more conservative amino acid substitutions, and the isolated or purified Ab, or synthetic or recombinant Ab, or Ag binding fragment thereof, or monomeric or dimeric ABP, retains its ability to specifically bind to a human homeobox NKX-2.1 protein or polypeptide; or
(e) the isolated or purified Ab, or synthetic or recombinant Ab, or Ag binding fragment thereof, or monomeric or dimeric ABP has two, three, four, five, six, seven, eight, nine, ten, eleven, twelve thirteen, fourteen or fifteen conservative amino acid substitutions, and the recombinant Ab, or Ag binding fragment thereof, or monomeric or dimeric ABP retains its ability to specifically bind to a human homeobox NKX-2.1 protein or polypeptide;
(f) the light chain variable region further comprises at least a portion of a light chain constant region;
(g) the light chain constant region comprises an amino acid sequence:
(SEQ ID NO: 8)
GDPVAPTVLIFPPAADQVATGTVTIVCVANKYFPDVTVTWEVDGTTQTT
GIENSKTPQNSADCTYNLSSTLTLTSTQYNSHKEYTCKVTQGTTSVVQS
FNRGDC;
(h) the heavy chain variable region further comprises at least a portion of a heavy chain constant region;
(i) the heavy chain constant region comprises an amino acid sequence:
(SEQ ID NO: 3)
GQPKAPSVFPLAPCCGDTPSSTVTLGCLVKGYLPEPVTVTWNSGT
LTNGVRTFPSVRQSSGLYSLSSVVSVTSSSQPVTCNVAHPATNTKVDKT
VAPSTCSKPTCPPPELLGGPSVFIFPPKPKDTLMISRTPEVTCVVVDVS
QDDPEVQFTWYINNEQVRTARPPLREQQFNSTIRVVSTLPIAHQDWLRG
KEFKCKVHNKALPAPIEKTISKARGQPLEPKVYTMGPPREELSSRSVSL
TCMINGFYPSDISVEWEKNGKAEDNYKTTPAVLDSDGSYFLYSKLSVPT
SEWQRGDVFTCSVMHEALHNHYTQKSISRSPGK;
(j) the light chain variable region further comprises at least a portion of a light chain constant region; and, the heavy chain variable region further comprises at least a portion of a heavy chain constant region; or
(k) the isolated or purified, or the recombinant antibody (Ab), or antigen binding fragment thereof, or monomeric or dimeric antigen binding protein, comprises:
a light chain comprising an amino acid sequence (SEQ ID NO:9); and
a heavy chain comprising an amino acid sequence (SEQ ID NO:4).

5. The isolated or purified Ab, or synthetic or recombinant Ab, or Ag binding fragment thereof, or monomeric or dimeric ABP, of claim 1, wherein:
(a) the heavy chain constant region comprises amino acid sequence from a IgG, IgM, IgA, IgD or IgE isotype, or the heavy chain constant region substantially comprises amino acid sequence from a IgG, IgM, IgA, IgD or IgE isotype;
(b) the light chain constant region comprises amino acid sequence from a kappa (κ) or lambda (λ) isotype, or the light chain constant region substantially comprises amino acid sequence from a kappa (κ) or lambda (λ) isotype;
(c) at least a portion of the heavy chain constant region, at least a portion of the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat or substantially derived from a human, a rabbit, a mouse or a rat;
(d) at least a portion of the heavy chain constant region, at least a portion of the light chain constant region, or at least a portion of the heavy chain constant region and the light chain constant region, is or comprises a synthetic amino acid sequence;
(e) the recombinant Ab, the Ag binding fragment thereof, or monomeric or dimeric ABP, or the heavy chain constant region, or the light chain constant region, or the heavy chain constant region and the light chain constant region, further comprises or is bound to a heterologous protein, peptide, or a compound or a composition;
(f) the heterologous protein or peptide, or the compound or a composition, comprises a detectable protein, a detectable agent or a binding moiety;
(g) the heterologous protein or peptide comprises a carrier protein;
(h) the heterologous protein, peptide or the compound or composition, is covalently conjugated to the recombinant antibody (Ab), or Ag binding fragment thereof, or monomeric or dimeric ABP;
(i) the detectable agent or binding moiety comprises a biotin, a fluorescent or chemiluminescent label, a fluorophore, perylene, fluorenyl, coumarin, 7-methoxycoumarin (Mca), 4-(dimethylaminoazo)benzene-4-carboxylic acid (dabcyl), Tamra, boron-dipyrromethene (BODIPY), or derivatives thereof, a dye, a radioisotope, a quantum dot or photoluminescent aqueous nanocrystal, a hapten, or an antibody binding epitope or domain;
(j) the dye is or comprises rhodamine, [2-(4-nitro-2,1,3-benzoxadiazol-7-yl)aminoethyl]trimethylammonium (NBD), nile red or nile blue, or is a fluorescent dye comprising sulfoindocyanine;
(l) the fluorophore is or comprises a dansyl, a fluorescein, a carboxyfluorescein (FAM) or a 6-FAM moiety;
(m) the dye is or comprises a cyanine dye, a Cy3 or a Cy5;
(n) the hapten is or comprises a biotin, a theophylline, a digoxigenin, a carborane, a fluorescein or a bromodeoxyuridine moiety; or,
(o) the Ab, or Ag binding fragment thereof, or monomeric or dimeric ABP is a recombinant Ab, or Ag binding fragment thereof, or monomeric or dimeric ABP, or comprises a peptide or polypeptide made by a recombinant technique.

6. A chimeric, synthetic or recombinant nucleic acid comprising: a nucleic acid sequence encoding a Ab, or Ag binding fragment thereof, or monomeric or dimeric ABP of claim 1.

7. The chimeric, synthetic or recombinant nucleic acid of claim 6, wherein:
(a) the chimeric or recombinant nucleic acid further comprises and is operatively linked to a transcriptional regulatory element;
(b) the transcriptional regulatory element comprises a promoter;
(c) the promoter is an inducible promoter or a constitutive promoter;
(d) the chimeric or recombinant nucleic acid further comprises sequence encoding an amino terminal signal peptide;
(e) the amino terminal signal peptide comprises the amino acid sequence
(SEQ ID NO: 5)
METGLRWLLLVAVLKGVQC;
(SEQ ID NO: 12)
MDTRAPTQLLGLLLLWLPGATF;
(f) the amino terminal signal peptide SEQ ID NO:5 is amino terminal to a VH chain SEQ ID NO:1, or is amino terminal to SEQ ID NO:4; or the amino terminal signal peptide SEQ ID NO:12 is amino terminal to a VL chain SEQ ID NO:2, or is amino terminal to SEQ ID NO:9.

8. An expression cassette, a vector, a recombinant virus, an artificial chromosome, a cosmid or a plasmid comprising a chimeric, synthetic or a recombinant nucleic acid of claim 6.

9. A cell comprising or having contained wherein:
(a) a chimeric, synthetic or recombinant antibody or dimeric antigen binding protein of claim 1,
(b) a chimeric or recombinant nucleic acid encoding the chimeric, synthetic or recombinant antibody or dimeric antigen binding protein of claim 1,
(c) an expression cassette, vector, recombinant virus, artificial chromosome, cosmid or plasmid of encoding the chimeric, synthetic or recombinant antibody or dimeric antigen binding protein of claim 1.

10. The cell of claim 9, wherein the cell is a bacterial, fungal, mammalian, yeast, insect or plant cell, or the mammalian cell is a human cell.

11. A method for detecting the presence of a human homeobox NKX-2.1 protein or polypeptide in or on a cell, a tissue, an organ or a portion of any of the foregoing, comprising:
(a) contacting the cell, tissue or organ or portion of any of the foregoing with at least one isolated or purified, or synthetic or recombinant Ab, or Ag binding fragment thereof, or monomeric or dimeric ABP of claim 1, and
(b) detecting the specific binding of the at least one Ab, or Ag binding fragment thereof, or monomeric or dimeric ABP with a human homeobox NKX-2.1 protein or polypeptide, in or on the cell, tissue or organ or portion of any of the foregoing,
thereby detecting the presence of the human homeobox NKX-2.1 protein or polypeptide in or on the cell, tissue, organ or portion of any of the foregoing.

12. The method of claim 11, wherein:
(a) the at least one Ab, or Ag binding fragment thereof, or monomeric or dimeric ABP comprises a variable heavy chain (VH) having an amino acid sequence comprising SEQ ID NO:1 and a variable light chain (VL) having an amino acid sequence comprising SEQ ID NO:2;
(b) the at least one Ab, or Ag binding fragment thereof, or monomeric or dimeric ABP comprises a variable heavy chain (VH) having an amino acid sequence comprising SEQ ID NO:4 and a variable light chain (VL) having an amino acid sequence comprising SEQ ID NO:9;
(c) the at least one Ab, or Ag binding fragment thereof, or monomeric or dimeric ABP comprises a variable heavy chain (VH) having an amino acid sequence comprising SEQ ID NO:7 and a variable light chain (VL) having an amino acid sequence comprising SEQ ID NO:11;
(d) the method comprises contacting the cell, tissue or organ or portion of any of the foregoing with two or at least two Abs, or Ag binding fragments thereof, or monomeric or dimeric ABPs, or a mixture of two or at least two different Abs, or Ag binding fragments thereof, or monomeric or dimeric ABPs, and wherein the two or at least two Abs, or Ag binding fragments thereof, or monomeric or dimeric ABPs or the two or at least two different Abs, or Ag binding fragments thereof, or monomeric or dimeric ABPs, comprise an Abs, or Ag binding fragments thereof, or monomeric or dimeric ABPs of claim 1;
(e) the method of (d), wherein the contacting comprises use of an immunohistochemistry (IHC) assay;
(f) the method of any of (a) to (d), further comprising contacting the Ab, or Ag binding fragment thereof, or monomeric or dimeric ABP, specifically bound to a human homeobox NKX-2.1 polypeptide or peptide, with a detectable agent to indicate or signal the specific binding of the Ab, or Ag binding fragment thereof, or monomeric or dimeric ABP, to the human homeobox NKX-2.1 polypeptide or peptide;
(g) the method of (f), wherein the detectable agent specifically binds to the Ab, or Ag binding fragment thereof, or monomeric or dimeric ABP;
(h) the cell, tissue, organ or a portion of any of the foregoing is or comprises or is derived from a cancer cell,
(i) wherein the cancer cell is a thyroid cancer cell, a lung cancer cell, or a carcinoma cell;
(j) the carcinoma cell is a large cell carcinoma, a small cell carcinoma, a lung carcinoma cell or an adenocarcinoma cell;
(k) the small cell carcinoma is a small cell lung carcinoma or an extrapulmonary small cell carcinoma, or the adenocarcinoma cell is a pulmonary adenocarcinoma cell or a thyroid adenocarcinoma cell;
(l) the cancer cell is a squamous carcinoma (SCC) cell, a leukemia cell, a carcinoid tumor, a mamma carcinoma, a colon carcinoma cell, a malignant melanoma cell or a multiple myeloma cell, a plasmacytoma cell or a lymphoma cell or a cancer derived from a lymphocyte;
(m) the lymphocyte is an early B cell, a pro-B cell, a pre-B lymphocyte, a mature B-lymphocyte, or a T lymphocyte or the T lymphocyte is a parafollicular T lymphocyte or a subpopulation of parafollicular T lymphocytes;
(n) the cell is a lung cell, a thyroid cell, a follicular center cell, or a cell in a tonsil, a lymph node germinal center, a bone marrow stem cell, a myelopoietic cell, a liver bile canalicular cell, a renal glomerular cell, a proximal tubular cell, a breast myoepithelial cell, a stromal cell around or associated with an infiltrating tumor cell, a kidney cell, an epithelial cell, a cerebellum cell, a prostate cell, a kidney cell, a pancreas cell or a bone marrow cell;
(o) the epithelial cell is or is derived from a brain, lung, intestine, kidney, breast or placental epithelial cell; the lung cell is a lung pneumocyte cell (and optionally the lung pneumocyte cell is a type II pneumocyte cell), or a Clara or nonciliated bronchiolar secretory cell; or the thyroid cell is a thyroid follicular cell or a thyroid parafollicular cell;
(p) the organ is a liver, prostate or lung;
(q) the cell, tissue, organ or a portion of any of the foregoing is or is derived from a lung adenocarcinoma; and/or
(r) the Ab, or Ag binding fragment thereof, or monomeric or dimeric ABP is less sensitive (or binds with lower affinity) in specific binding to lung squamous cell carcinoma cells than specifically binding to adenocarcinoma cells, thereby resulting in fewer lung squamous cell carcinoma false positives and better positive identification of lung adenocarcinoma.

13. A method for detecting or diagnosing a cancer,
wherein the method comprises detecting expression or presence of a human homeobox NKX-2.1 polypeptide or peptide in or on a cell, tissue or organ sample using a method of claim 11,
wherein the detecting of the specific binding of the Ab, or Ag binding fragment thereof, or monomeric or dimeric ABP with a human homeobox NKX-2.1 polypeptide or peptide in or on the cell, tissue or organ or portion of any of the foregoing, detects or diagnoses, or assists in the detection or diagnosis of, the cancer.

14. The method of claim 13, wherein:
(a) the cancer is a thyroid cancer cell, a lung cancer cell, or a carcinoma cell,
(b) the cancer is a carcinoma cell, or a large cell carcinoma, a small cell carcinoma (optionally a small cell lung carcinoma or an extrapulmonary small cell carcinoma), a lung carcinoma cell or an adenocarcinoma cell, and optionally the adenocarcinoma cell is a pulmonary adenocarcinoma cell or a thyroid adenocarcinoma cell),
(c) the cancer is a squamous carcinoma (SCC) cell, a leukemia cell, a carcinoid tumor, a mamma carcinoma, a colon carcinoma cell, a malignant melanoma cell or a multiple myeloma cell, a plasmacytoma cell or a lymphoma cell or a cancer derived from a lymphocyte, wherein optionally the lymphocyte is an early B cell, a pro-B cell, a pre-B lymphocyte, a mature B-lymphocyte, a T lymphocyte, a parafollicular T lymphocyte, a cell derived from a subpopulation of parafollicular T lymphocytes;
(d) the cancer is a lung carcinoma, or a lung adenocarcinoma,
(e) the Ab, or Ag binding fragment thereof, or monomeric or dimeric ABP is less sensitive (or binds with lower affinity) in specific binding to lung squamous cell carcinoma cells than specifically binding to adenocarcinoma cells, thereby resulting in fewer lung squamous cell carcinoma false positives and better positive identification of lung adenocarcinoma;
(f) the cell, tissue or organ sample is from an individual in need thereof;
(g) the detection comprises conducting an immunohistochemistry (IHC) assay,
(h) at least two Abs, or Ag binding fragments thereof, or monomeric or dimeric ABPs, are used to contact the cell, tissue or organ sample; or
(i) one of the two Abs, or Ag binding fragments thereof, or monomeric or dimeric ABPs comprises a variable heavy chain (VH) having an amino acid sequence comprising SEQ ID NO:1 and a variable light chain (VL) having an amino acid sequence comprising SEQ ID NO:2.

15. A method for treating, ameliorating or preventing a cancer comprising first detecting or diagnosing the cancer using the method of claim 13, followed by treatment of the individual in need thereof for the treatment, amelioration or prevention of the cancer.

16. The method of claim 15, wherein:
(a) the cancer a thyroid cancer cell, a lung cancer cell, or a carcinoma cell,
(b) the cancer cell is a carcinoma cell, or a large cell carcinoma, a small cell carcinoma (optionally a small cell lung carcinoma or an extrapulmonary small cell carcinoma), a lung carcinoma cell or an adenocarcinoma cell, and optionally the adenocarcinoma cell is a pulmonary adenocarcinoma cell or a thyroid adenocarcinoma cell),
(c) the cancer cell is a squamous carcinoma (SCC) cell, a leukemia cell, a carcinoid tumor, a mamma carcinoma, a colon carcinoma cell, a malignant melanoma cell or a multiple myeloma cell, a plasmacytoma cell or a lymphoma cell or a cancer derived from a lymphocyte,
(d) the lymphocyte is an early B cell, a pro-B cell, a pre-B lymphocyte, a mature B-lymphocyte, a T lymphocyte, a parafollicular T lymphocyte, a cell derived from a subpopulation of parafollicular T lymphocytes,
(e) the carcinoma is a lung carcinoma, or the lung carcinoma is a lung adenocarcinoma, or
(f) the Ab, or Ag binding fragment thereof, or monomeric or dimeric ABP is less sensitive (or binds with lower affinity) in specific binding to lung squamous cell carcinoma cells than specifically binding to lung adenocarcinoma cells, thereby resulting in fewer lung squamous cell carcinoma false positives and better positive identification of lung adenocarcinoma.

17. (canceled)

18. (canceled)

19. An isolated or purified, or synthetic or recombinant antibody (Ab), or antigen (Ag) binding fragment thereof, or monomeric or dimeric antigen binding protein (ABP) of claim 1, for use in detecting or diagnosing a cancer, or treating, ameliorating or preventing a cancer.

20. The isolated or purified, or synthetic or recombinant antibody (Ab), or antigen (Ag) binding fragment thereof, or monomeric or dimeric antigen binding protein (ABP) of claim 19, wherein:
(a) the cancer is a thyroid cancer cell, a lung cancer cell, or a carcinoma cell,
(b) the cancer is a carcinoma cell, or a large cell carcinoma, a small cell carcinoma (optionally a small cell lung carcinoma or an extrapulmonary small cell carcinoma), a lung carcinoma cell or an adenocarcinoma cell, and optionally the adenocarcinoma cell is a pulmonary adenocarcinoma cell or a thyroid adenocarcinoma cell),
(c) the cancer cell is a squamous carcinoma (SCC) cell, a leukemia cell, a carcinoid tumor, a mamma carcinoma, a colon carcinoma cell, a malignant melanoma cell or a multiple myeloma cell, a plasmacytoma cell or a lymphoma cell or a cancer derived from a lymphocyte,
(d) the lymphocyte is an early B cell, a pro-B cell, a pre-B lymphocyte, a mature B-lymphocyte, a T lymphocyte, a parafollicular T lymphocyte, a cell derived from a subpopulation of parafollicular T lymphocytes,
(e) the carcinoma is a lung carcinoma, or the lung carcinoma is a lung adenocarcinoma,
(f) the Ab, or Ag binding fragment thereof, or monomeric or dimeric ABP is less sensitive (or binds with lower affinity) in specific binding to lung squamous cell carcinoma cells than specifically binding to adenocarcinoma cells, thereby resulting in fewer lung squamous cell carcinoma false positives and better positive identification of lung adenocarcinoma, or
(g) the detecting or diagnosing comprises conducting an immunohistochemistry (IHC) assay.

21. A kit comprising:
(a) a isolated or purified, or synthetic or chimeric or recombinant antibody of claim 1; a chimeric or a recombinant nucleic acid encoding the synthetic or chimeric or recombinant antibody; or an expression cassette, vector, recombinant virus, artificial chromosome, cosmid or plasmid encoding the synthetic or chimeric or recombinant antibody; or, a cell containing the synthetic or chimeric or recombinant antibody or the chimeric or the recombinant nucleic acid, or the expression cassette, vector, recombinant virus, artificial chromosome, cosmid or plasmid; and/or
(b) components needed for an immunohistochemistry (IHC) assay, and/or comprises instructions for practicing a method with synthetic or chimeric or recombinant antibody.

22. An isolated or purified antibody (Ab), or synthetic or recombinant Ab, or antigen (Ag) binding fragment thereof comprising:
(a) a heavy chain variable region (VH) capable of specifically binding to a human homeobox NKX-2.1 protein or polypeptide, comprising an amino acid sequence comprising the three CDR1, CDR2 and CDR3 complementarity determining regions (CDRs) of SEQ ID NO:1, or CDR1 amino acid (aa) residues GFSLSSYA (residues 25 to 32 of SEQ ID NO:1), CDR2 aa residues INSYGNA (residues 50 to 56 of SEQ ID NO:1), and CDR3 aa residues TTTHSYGFGAFDP (residues 95 to 107 of SEQ ID NO:1); and
(b) a light chain variable region (VL) capable of specifically bind to a human homeobox NKX-2.1 protein or polypeptide comprising an amino acid sequence comprising the three CDR1, CDR2 and CDR3 complementarity determining regions (CDRs) of SEQ ID NO:2, or CDR1 amino acid (aa) residues ESAYNNR (residues 27 to 33 of SEQ ID NO:2), CDR2 aa residues DAS (residues 51 to 53 of SEQ ID NO:2), and CDR3 aa residues QGVYSSVSDVYA (residues 90 to 101 of SEQ ID NO:2).

23. The isolated or purified antibody (Ab), or synthetic or recombinant Ab, or antigen (Ag) binding fragment thereof of claim 22, wherein the heavy chain variable region comprises SEQ ID NO: 1 and the light chain variable region comprises SEQ ID NO:2.
